(19) United States
(12) Patent Application Publication
(10) Pub. No.: US 2003/0192065 A1
(43) Pub. Date: Oct. 9, 2003

(54) HUMAN NERVE GROWTH FACTOR EXON 1 AND EXON 3 PROMOTERS

(76) Inventors: MATTHEW D. LINNIK, SOLANA BEACH, CA (US); MARGARET M. RACKE, CINCINNATI, OH (US); JOAN M. KRAKOWSKY, BASKING RIDGE, NJ (US); ARUN SUBRAMANIAM, LEBANON, NJ (US)

Correspondence Address: ROSS J. OEHLER, AVENTIS PHARMACEUTICALS INC., ROUTE 202-206, MAIL CODE: D303A, BRIDGEWATER, NJ, 08807, US

(21) Appl. No.: 09/326,885

(22) Filed: Jun. 7, 1999

Related U.S. Application Data
(62) Division of application No. 09/020,179, filed on Feb. 6, 1998, now abandoned.

(51) Int. Cl. A01K 67/00; C12Q 1/68; C07H 21/04; C12P 21/02; C12N 5/06; C07K 14/48

(52) U.S. Cl. 800/8; 435/69.1; 435/320.1; 435/325; 530/399; 536/23.5; 435/6

(57) ABSTRACT

Novel human nerve growth factor exon 1 promoter, human nerve growth factor exon 3 promoter, fragments thereof, and modified forms thereof are described. The invention is also directed to vectors containing such promoters, cells transformed with the same, including animal models and transgenic animals containing such sequence and assay methods using these promoters.

Several lines of evidence point to the potential therapeutic utility of nerve growth factor in neurodegenerative diseases. NGF has been shown to prevent neurons from dying after experimentally induced injuries including ischemia (Shigeno T, et al., J Neurosci 11:2914-2919, 1991; Yamamoto S, et. al, Neurosci Lett 141:161-165, 1992; Pechan P A, et al., NeuroReport 6:669-672, 1995; Holtzman D M, et al., Ann Neurol 39:114-122, 1996), concussion (Hayes R L, et al., J Neurotrauma 12:933-41, 1995; Sinson G, et al., J Neurochem 65:2209-2216, 1995), and axotomy (Williams M and Braunwalder A., J Neurochem 47:88-97, 1986; Kromer, L. F. Science 235:214-216, 1987). NGF can also help to sustain function in aged or damaged neurons by maintaining neuronal phenotype and inducing neurite outgrowth (Fischer W, et al., Nature 329:65-68, 1987; Fischer W, et al, J Neurosci 11:1889-1906, 1991; Rylett, R. J., et al., J Neurosci 13:3956-3963, 1993; Chen K S, et al., Neuroscience 68(1): 19-27, 1995; Tuszynski M H and Gage F H, Mol Neurobiol 10:151-167, 1995).

Systemic administration of NGF is an inefficient method to achieve brain exposure due to the limited ability of NGF to cross the blood-brain barrier (Poduslo J F and Curran G L, Molec Brain Res 36:280-286, 1996). Several alternative routes of administration have proven effective, including direct intracerebroventricular administration, implantation of producer cell lines (Rosenberg M B, et al., Science 242:1575-1578, 1988), conjugation to actively transported molecules (Friden P M, et al., Science 259:373-377, 1993; Kordower J H, et al., PNAS USA September 13; 91(19): 9077-80, 1994) and transcriptional upregulation by low molecular weight compounds.

A number of small molecules have been identified that increase NGF mRNA transcription (Mocchetti I, Ann Rev Pharmacol Tioxicol 32:303-328, 1991; Carswell S, Exp Neurol 124:36-42, 1993) and some of these compounds have been demonstrated to mimic the pharmacological action of exogenous NGF in vivo (Lee, T.-H., et al., Stroke 25:1425-1432, 1994; Kaechi K, et al., JPET 264(1): 321-6, 1993; Kaechi K, et al., JPET 272:1300-1304, 1995). The majority of NGF-inducing compounds have been shown to upregulate NGF mRNA transcription via the two promoter regions which have been identified in the mouse NGF gene (Selby M J, et al., Molec Cell Biol 7:3057-3064, 1987; Nitta A., et al., Eur J Pharmacol 250:23-30, 1993). Recently, a third promoter has been suggested in the rat NGF gene (Timmusk T, et al., Soc Neurosci Absts 21:33, 1995).

The mouse promoter at exon 1 has been well studied and a functional AP-1 regulatory element has been described 35 bases 3′ of the start of exon 1 (D'Mello S R, and Heinrich G. J Neurochem 57:1570-1576, 1991; D'Mello S R, and Heinrich G., Molec Cell Neurosci 2:157-167, 1991; Cowie A, et al., Mol Brain Res 27:58-62, 1994). An identical element exists in the human gene at the same location (Cartwright M, et al., Mol Brain Res 15:67-75, 1992). However, the regulation of the human and mouse NGF promoters is not identical. For example, functional analyses of the human gene revealed a 5′ consensus AP-1 site at −74 in the human gene that is not present in the mouse gene (Cartwright M, et al., Mol Brain Res 15:67-75, 1992).

The importance of 5′ sequence of exon 1 in basal expression also depends on the nature of the reporter vector. Large differences in basal transcription were reported in cells containing various 5′ ends when using human growth hormone as a reporter system (D'Mello S R, and Heinrich G., Molec Cell Neurosci 2:157-167, 1991; Cowie A, et al., Mol Brain Res 27:58-62, 1994). However, Cowie et al. (Cowie A, et al., Mol Brain Res 27:58-62, 1994) present evidence that the length of the 5′ end has a minimal effect when using a different reporter system.

The 3′ intron 1 AP-1 site is present in humans and rodents and is also thought to be involved in basal expression, lesion induced increases in NGF mRNA and phorbol ester responsiveness (D'Mello S R, and Heinrich G., Molec Cell Neurosci 2:157-167, 1991; Cowie A, et al., Mol Brain Res 27:58-62, 1994; Hengerer B, et al., Proc. Natl. Acad. Sci. USA 87:3899-3903 (1990).

The pharmacological regulation of NGF gene expression is also sensitive to the transcriptional environment. For example, phorbol 12-myristate 13-acetate (PMA) enhances the synthesis of NGF in mouse L929 fibroblasts and in primary glial cells (D'Mello S R, and Heinrich G. J Neurochem 55:718-721, 1990; Wion D, et al., FEBS Lett 262:42-44, 1990; Neveu I, et al., Brain Res 570:316-322, 1992) but suppresses expression in ROS 17/2.8 osteoblastic cells (Jehan F, et al., Molec and Cell Endocrinol 116:149-156, 1996). Several recent reports have identified astrocytes as a source of NGF in vivo, particularly after a traumatic insult. (Lee T H, et al., Brain Res 713:199-210, 1996; Kossmann T, et al., Brain Res 713:143-152, 1996; DeKosky S T, et al., Ann Neurol 39:123-7, 1996) and it has been recognized that glial derived cell lines can synthesize and secrete nerve growth factor (Carman-Krzan M, et al, J-Neurochem 56(2): 636-43, 1991; Lu B, et al., J-Neurosci 11(2): 318-26, 1991).

The majority of pharmacological studies on the NGF promoter have been conducted with the rodent gene which is homologous but not identical to the human gene. The human gene structure is not yet completely known. The human regions corresponding to exons 3 and 4 of the mouse gene have been described (Ullrich A, et al., Nature 303:821-825, 1983), as well as a cDNA including exon 1b which corresponds to transcript (B) in the mouse (Selby M J, et al., Molec Cell Biol 7:3057-3064, 1987; Borsani G, et al., Nuc. Acids Res 18:4020, 1990).

A number of physiologic changes are known to induce NGF in vivo. A sciatic nerve lesion induces NGF in normeuronal cells of the sciatic nerve (Lincholm, D. R., et al, Nature 350:658-659 (1987). Transection of fimbria fornix induces NGF in the hippocampus and basal forebrain. (Gasser, U. E., et al., Brain Res. 376:351-356, 1986, Weskamp, G., et al., Neurosci. Lett. 70:121-126, 1986). Electrolytic lesion of the septohippocampal pathway induces NGF in the hippocampus and basal forebrain astrocytes. (Oderfeld-Nowak, G., et al., Neurochem. Int. 21:455-461, 1992). Needle injection into rat hippocampus induces NGF in the cortex and hippocampus. (Ballarin, M., et al., Exp. Neurol., 114:35-43, 1991). Denervation of niagral dopaminergic cells induces NGF in the cortex and hippocampus. (Nitta, A., et al., Neurosci. Lett. 144:152-156, 1992). Limbic seizures induce NGF in hippocampal, cortical and olfactory neurons. (Gall, C. M and Issackson, P. J., Science, 245:758-761, 1986). Transection of the optic nerve induces NGF in the glia cells of the optic nerve. (Lu, B., et al., J. Neurosci., 11:318-326, 1991). Excitotoxic destruction of hippocampal neurons induces NGF in hippocampal glia. (Bakhit, C., et al., Brain Res. 560:76-83, 1991). Bilateral decortation induces NGF in the glia cells in the basal forebrain and neostriatum. (Lorex, H. P., et al, Brain Res. 454:355-360, 1988). Finally, evoking aggressive behavior in adult males is shown to induce NGF in male mouse hypothalamus. (Psillantini, M. G., et al., Proc. Natl. Acad. Sci. USA 86:8555-8559, 1989).

Seizure activity has been shown to transiently increase mRNA levels of NGF and other neurotrophic factors, such as BDNF, in cortical and hippocampal neurons. These changes are observed after limbic seizures have been induced by a wide variety of insults, such as dentate hilar lesion, kainic acid, or kindling, as well as after injections of bicuculline or pentylenetratrazol. (Lindvall, O., et al., TINS 17(11) 1994:490-496).

Alzheimer's disease is a neurodegenerative disease that is partially characterized by progressive loss of cognitive function. Biological changes associated with Alzheimer's disease include formation of amyloid-rich neutic plaques and neurofibrillary tangles in areas associated with learning and memory—the hippocampus and neocortex. Acetylcholine-containing (cholinergic) neurons found in the basal forebrain decrease, and the severity of the cognitive deficit observed in Alzheimer's patients closely correlates with the loss of cholinergic neurons in the basal forebrain.

High levels of NGF protein and mRNA encoding NGF are localized in the hippocampus and neocortex, the major cholinergic target areas of the basal forebrain neurons. These cholinergic neurons have been shown to shrink and die following damage and with age, possibly due to a loss of target contact with the hippocampus and cortex.

Exogenous administration of NGF into the CNS increases the survival, function and potentially the regeneration of damaged and aged hippocampal and cortical neurons in rodents and nonhuman primates. These studies support the role of administering NGF or increasing local NGF levels, to prevent the cholinergic degeneration observed in Alzheimer's patients and potentially induce neurite outgrowth in surviving neurons.

Delivery of exogenous NGF presents some particular challenges. If administered intravenously, NGF is not able to cross the blood-brain barrier and hence is not able to get to the target neurons of the hippocampus or cortex. Administration directly into the brain, via a ventricular reservoir or pump, is costly, difficult and exposes the central nervous system to potential infections, as well as being uncomfortable for the patient.

A possible solution to delivery problems may be bioactive fragments of NGF, which may have a higher degree of biological activity than NGF and more easily penetrate the blood-brain barrier. Smaller fragments may also be more cost effective, as they are smaller and easier to prepare recombinantly. However, to date, truncated NGF fragments have not been successfully administered and appear to lose activity.

Another possible solution is implantation of NGF-producing cell lines directly into the site of needed activity. However, this approach requires genetic manipulation of a cell, which may present significant regulatory approval problems. Many of the host cell lines used, e.g., fibroblasts, are possibly tumorgenic and may continue to proliferate after transplantation into the CNS. In addition, cell surface markers on the cell line may provoke rejection by the immune system. It is not currently possible to control the level of NGF secretion into the adjacent tissue.

Another potential therapeutic approach is upregulation of endogenous NGF production by administration of a small molecule which directly activates transcription of NGF and hence leads to greater NGF mRNA and ultimately increased NGF protein production. Generally, small molecules are capable of passing through the blood-brain barrier, and may easily be formulated for either intravenous or oral administration.

The present invention is directed to the novel human genomic DNA sequences adjacent to, or within, the NGF gene which contain promoters for NGF transcription. Using the present sequences, reporter constructs comprising all or part of the DNA sequence provided herein attached to a reporter gene, for example, the luciferase gene, β-galactosidase or green fluorescent protein (GFP), may be prepared. These novel reporter constructs may be then used to screen compounds for their ability to affect transcription of NGF. The present invention is also directed to a method for assaying a compound for its ability to affect transcription of the NGF promoter. Preferred embodiments of nucleic acid of the invention are as follows:

1. An isolated nucleic acid comprising human nerve growth factor exon 1 promoter selected from 1-1786, 2274-2846, human nerve growth factor exon 3 promoter 1-1877, fragment thereof, or modified form thereof.

2 The nucleic acid according to 1, wherein the nucleic acid is nerve growth factor exon 1 promoter, fragment thereof, or modified form thereof.

3 The nucleic acid according to 2, wherein the nucleic acid is human nerve growth factor exon 1 promoter 1-1786, fragment thereof, or modified form thereof.

4 The nucleic acid according to 2, wherein the nucleic acid is human nerve growth factor exon 1 promoter to 2274-2846, fragment thereof, or modified form thereof.

5 The nucleic acid according to 1, wherein the nucleic acid is human nerve growth factor exon 3 promoter 1-1877, fragment thereof, or modified form thereof.

6 The nucleic acid according to 1, wherein the nucleic acid comprises a consensus binding motif from human nerve growth factor exon 1 promoter selected from 1-1786, 2274-2846, human nerve growth factor exon 3 promoter 11-1877, or modified form thereof.

7 The nucleic acid according to 6, wherein the nucleic acid comprises a consensus binding motif.

8. The nucleic acid according to 7, wherein the consensus binding motif comprises a CAAT box or TATA box.

9. The nucleic acid according to 6, wherein the consensus binding motif is binding site for a ribosome.

10. The nucleic acid according to 7, wherein the consensus binding motif is selected from the group consisting of NF-Ytk, NF-Y MCHII, AABS, ATF, Ad2MLP, EGR-1, ELP RS, GCN4 HIS3.1, GCN4 HIS4.3, GCN4 HIS4.4, GCRE, OBF H2B1, OBF histone, NF E1.3, NF E1.6 and NF E1.5.

11. The nucleic acid according to claim 6, wherein the consensus binding motif is selected from the group consisting of AP1, AP2, AP3, AP4, AP5, E4TF1, CTF/NF-1, NF-KB, TFIID, TFIIIA, p53, GM-CSF or NF IL-6.

12. The nucleic acid according to 1, wherein the nucleic acid comprises a consensus binding motif of a transcription factor in an inflammatory pathway.

13. The nucleic acid according to 1, wherein the nucleic acid comprises a consensus binding motif of a transcription factor in a cell-death pathway.

14. The nucleic acid according to 1, wherein the nucleic acid comprises a consensus binding motif of a transcription factor in a tumorgenic pathway.

15. The nucleic acid according to 1, wherein the nucleic acid comprises an enhancer sequence, repressor sequence or consensus binding motif for a transcription activating factor.

16. The nucleic acid according to 1, wherein the nucleic acid comprises a natural or a modified derivative of deoxyribonucleic acid or ribonucleic acid.

17. The nucleic acid according to 12, wherein the nucleic acid comprises a phosphodiester, methylphosphonate, phosphoramidate, isopropyl phosphate triester, phosphorothioate, phosphothionate, phosphotriester or boranophosphate.

The present invention is also directed to manipulation of the human NGF exon 1 promoter, exon 3 promoter, fragment thereof, or modified form thereof, plasmids resulting from such manipulation and cells transformed or transfected with such plasmids and transgenic animals containing such plasmids. The invention includes manipulation where exogenous promoters are inserted into human NGF exon 1 promoter or exon 3 promoter by, e.g., homologous recombination. The invention also includes manipulation where all or part of a human exon 1 promoter or exon 3 promoter is replaced by a nonnaturally-occurring exogenous or otherwise endogenous DNA, which may be DNA from another gene, e.g., intron or exon of a gene other than NGF, from another chromosome, or a naturally-occurring variant of the human NGF exon 1 promoter or exon 3 promoter. An example of an endogenous modification of human NGF exon 3 promoter would be e.g., part or all of human NGF exon 1 promoter replacing part or all of human NGF exon 3 promoter. Similarly, this manipulation includes where a nonnaturally occurring exogenous or otherwise endogenous DNA encoding consensus binding motif replaces, is inserted or is deleted from the naturally occurring consensus binding motif, e.g., where the consensus binding motif of AP3, which is the consensus binding motif for protein kinase C responsive element in human NGF exon 3, e.g., starting at +116, −1608 or +2472, is replaced with, for example, PRL, the prolactin gene regulatory control element at −159 of human NGF exon 3, deletion or alteration of a CAAT box or TATA box located, for example, in human NGF exon 3 promoter or a regulatory control element from another gene, or may even be a synthetically-derived control element based on a consensus sequence. Alternatively, the invention is directed to insertion of regulatory elements, such as insertion of a CAAT box or TATA box in a non-naturally occurring site within human NGF exon 1 promoter or exon 3 promoter. Such manipulation may be accomplished by, for example, homologous recombination or site directed mutagenesis.

The present invention is also directed to modifications of human NGF exon 1 promoter or exon 3 promoter which modify transcription of human NGF. An example of such modification includes alteration of one or more lariat site in the human NGF exon 1 promoter or exon 3 promoter. A lariat site is a loosely palindromic sequence which permits the DNA to loop back on itself. Alteration of a lariat site may influence binding of transcription factors, even if the underlying consensus binding motif the transcription factor normally binds to is not altered. Another example of such modification is alteration of a splice donor site or splice acceptor site.

The present invention is also directed to constructs resulting from such above manipulation, plasmids and vectors containing such constructs, and cells containing such constructs. Specifically included within the present invention are genetically altered cells suitable for autologous transplantation, whereby human cells are manipulated to alter the naturally occurring NGF exon 1 promoter or exon 3 promoter to alter one, or more, naturally occurring consensus binding motif, add one, or more, non-naturally occurring consensus binding motif or delete, one or more, naturally occurring consensus binding motif, or other modifications of human NGF exon 1 promoter and/or exon 3 promoter.

The present invention is also directed to vectors comprising human NGF exon 1 promoter or exon 3 promoter, fragment thereof, or modifications thereof. The present vectors include expression vectors, such as a vector comprising the human NGF exon 1 promoter or exon 3 promoter, fragment thereof or modification thereof, and a marker gene, such as a gene encoding a detectable protein or conferring an altered, or detectable, phenotype or genotype. Especially preferred detectable proteins are reporter genes, and include luciferase, β-galactosidase, placental alkaline phosphatase and green fluorescent protein (GFP). The present invention is also directed to reporter vectors, which comprise an insertional site for a gene of interest and the gene encoding neomycin resistance under control of a thymidine kinase promoter. The present invention includes transformation vectors, such as a vector comprising the human NGF exon 1 promoter or exon 3 promoter, fragment thereof or modification thereof, and suitable for transfecting or transforming a suitable host cell. Examples of suitable transformation vectors include plasmids pGL, pGEM and phages, such as gt 10 and gt 11.

Especially preferred vectors are defective viral vectors, including amplicons. Defective viral vectors may result from one or more defective subgenomic viral particle(s) which contain an essential portion of the genome and require complementation of homologous “helper” virus for replication. Such defective viruses occur naturally and are also called defective interfering viruses (or D1 particles). D1 particles occur as RNA or DNA viruses, and have been identified in herpes viruses, including HSV, human cytomegalovirus, equine herpes virus. Especially preferred defective viral vectors of the present invention include amplicons comprising the human NGF exon 1 promoter or exon 3 promoter, fragment thereof or modification thereof. Preferred embodiments of vectors of the invention are as follows:

1. A vector comprising a nucleic acid human nerve growth factor exon 1 promoter 1-1786, 2274-2846, human nerve growth factor exon 3 promoter 1-1877, fragment thereof, or modified form thereof.

2 The vector according to 1, wherein the nucleic acid is nerve growth factor exon 1 promoter, fragment thereof, or modified form thereof.

3 The vector according to 2, wherein the nucleic acid is human nerve growth factor exon 1 promoter 1-1786, fragment thereof, or modified form thereof.

4 The vector according to 2, wherein the nucleic acid is human nerve growth factor exon 1 promoter to 2274-2846, fragment thereof, or modified form thereof.

5 The vector according to 1, wherein the nucleic acid is human nerve growth factor exon 3 promoter 1-1877, fragment thereof, or modified form thereof.

6 The vector according to 1, wherein the nucleic acid comprises a consensus binding motif from human nerve growth factor exon 1 promoter 1-1786, 2274-2846, human nerve growth factor exon 3 promoter 1-1877, fragment thereof, or modified form thereof.

7. The vector according to 6, wherein the nucleic acid comprises a consensus binding motif is selected from the group consisting of AP1, AP2, AP3, AP4, AP5, E4TF1, CTF/NF-1, NF-KB, TFIID, TFIIIA, p53, GM-CSF or NF IL-6.

8. The vector according to 6, wherein the consensus binding motif comprises a CAAT box or TATA box.

9. The vector according to 1, wherein the nucleic acid comprises an enhancer sequence, repressor sequence or consensus binding motif for a transcription factor.

10. The vector according to 1, wherein the nucleic acid comprises a consensus binding motif of a transcription factor in an inflammatory pathway.

11. The vector according to 1, wherein the nucleic acid comprises a consensus binding motif of a transcription factor in a cell-death pathway.

12. The vector according to 1, wherein the nucleic acid comprises a consensus binding motif of a transcription factor in a tumorgenic pathway.

13 The vector according to 1, wherein the vector is an amplicon, transcription vector, expression vector, reporter vector, insertion vector, replacement vector, or mutagenesis vector.

14 The vector according to 13, wherein the vector is pGL2 enhancer, pGL3 Basic or pGL3 neo.

15. The vector according to 13, wherein the amplicon provides a viral packaging system for cellular expression.

16. The vector according to 13, wherein the vector comprises a viral packaging system.

17. The vector according to 16, wherein the viral packaging system is a retrovirus, adenovirus, adeno-associated virus, or herpes virus system.

The present invention is also direct to a novel vector designed to incorporate the human NGF exon 1 promoter, exon 3 promoter, fragment thereof, or modification thereof. The vector comprises both a reporter gene and gene encoding antimetabolite resistance. The present invention is also directed to cells comprising such vectors, methods of assaying compounds using the same, and methods for identifying a compound capable of modifying transcription of a nucleic acid. Specific embodiments of the present invention are as follows:

1. A vector comprising pGL3-neo.

2. The vector according to 1, comprising a promoter sequence greater than 2 kilobases.

3. The vector according to 2, wherein the promoter is greater than 3 kilobases.

4. The vector according to 3, wherein the promoter is greater than 4 kilobases.

5. The vector according to 1, comprising a nucleic acid encoding human nerve growth factor exon 1 promoter 1-1786, 2274-2846, human nerve growth factor exon 3 promoter 1-1877, fragment thereof, or modified form thereof.

6. The vector according to claim 5, wherein the nucleic acid comprises human nerve growth factor exon 1 promoter 1-1786, 2274-2846, fragment thereof, or modified form thereof.

7. The vector according to 5, wherein the nucleic acid comprises human nerve growth factor exon 1 promoter 1-1786, fragment thereof, or modified form thereof.

8. The vector according to claim 5, wherein the nucleic acid comprises human nerve growth factor exon 1 promoter 2274-2846, fragment thereof, or modified form thereof.

9. The vector according to claim 1, wherein the nucleic acid comprises human nerve growth factor exon 3 promoter 1-1877, fragment thereof, or modified form thereof.

10. A cell comprising a vector according to 1.

11. A cell according to 10, wherein the cell is an animal cell.

12. A cell according to 11, wherein the cell is a human or primate cell.

13. A cell according to 12, wherein the cell is a human cell.

14. A cell according to 10, wherein the c ell is a yeast or bacterial cell.

15. An assay comprising a cell according to 10.

16. The assay according to 15, wherein the cell is human.

17. The assay according to 15, wherein the assay is suitable for high throughput screening.

18. The assay according to 15, wherein the assay permits simultaneous evaluation of multiple compounds.

19. The assay according to 15, wherein the assay is partially or fully automated.

20. A method for identifying a compound capable of modifying transcription of a nucleic acid, comprising contacting a compound with a cell according to 1.

The present invention may also be used in recombinant technology to produce proteins. Therefore, the present invention is directed to vectors wherein the human NGF exon 1 promoter or exon 3 promoter, fragment thereof, or modified form thereof, is operably linked to a gene encoding a protein and cells containing such vectors. The invention is also directed to methods of producing protein using the human NGF exon 1 promoter, or exon 3 promoter, fragment thereof, or modified form thereof. Preferred embodiments of the invention include the following:

1. A method of producing a protein comprising expressing a vector comprising a human nerve growth factor exon 1 promoter 1-1786, 2274-2846, human nerve growth factor exon 3 promoter 1-1877, fragment thereof, or modified form thereof operably linked to a gene encoding a protein.

2. The method according to 1, wherein the promoter comprises a human nerve growth factor exon 1 promoter 1-1786, 2274-2846, fragment thereof, or modified form thereof.

3. The method according to 2, wherein the promoter comprises a human nerve growth factor exon 1 promoter selected from 1-1786, fragment thereof, or modified form thereof

4. The method according to 2, wherein the promoter comprises a human nerve growth factor exon 1 promoter selected from 2274-2846, fragment thereof, or modified form thereof.

5. The method according to 1, wherein the promoter comprises a human nerve growth factor exon 3 promoter 1-1877, fragment thereof, or modified form thereof.

6. The method according to 1, wherein the vector comprises a consensus binding motif from human nerve growth factor exon 1 promoter 1-1786, 2274-2846, human nerve growth factor exon 3 promoter 1-1877, fragment thereof, or modified form thereof.

7. The method according to 1, wherein the promoter is operably linked to a gene encoding a selectable protein.

8. The method according to 7, wherein the selectable protein confers antimicrobial resistance.

9. The method according to 8, wherein the antimicrobial resistance is to neomycin, sulfonamide, penicillin, cephalosporin, aminoglycoside, tetracyclin, or modified forms thereof.

10. The method according to 1, wherein the protein is a naturally occurring mammalian neurotrophic factor or a modified naturally occurring mammalian neurotrophic factor.

11. The method according to 10, wherein the protein is a naturally occurring mammalian neurotrophic factor.

12. The method according to 11, wherein the protein is nerve growth factor.

13. The method according to 12, wherein the nerve growth factor is human.

14. The method according to 10, wherein the protein is a modified naturally occurring mammalian neurotrophic factor.

15. The method according to 14, wherein the protein is nerve growth factor.

16. The method according to 15, wherein the nerve growth factor is human.

The present invention also includes oligonucleotides encoding human NGF exon 1 promoter or exon 3 promoter, fragment thereof, or modified form thereof. Preferred oligonucleotides are antisense oligonucleotides to a fragment of either human NGF exon 1 promoter or exon 3 promoter. More preferred antisense oligonucleotides are to all or part of a consensus binding motif within either human NGF exon 1 promoter or exon 3 promoter.

Preferred oligonucleotides are about six to about one hundred bases long. Preferred antisense oligonucleotides are six to one hundred bases long, more preferred antisense oligonucleotides are about six to about fifty bases long, and even more preferred antisense oligonucleotides are about ten to about twenty five bases long. Especially preferred antisense oligonucleotides are about fifteen bases long.

Nucleic acid of the present invention may contain naturally occurring nucleotides or analogs thereof. Preferred naturally-occurring nucleotides are either deoxyribonucleic acid or ribonucleic acid. Preferred analogs of naturally-occurring nucleotides are modified phosphotriesters, bases or sugars. Especially preferred are phosphodiesters, methylphosphonates, phosphoramidates, isopropyl phosphate triesters, phosphorothioates, phosphothionates, phosphotriesters or boranophosphates.

The present invention includes methods of modifying regulation of human nerve growth factor by administration of an oligonucleotide encoding human NGF exon 1 promoter or exon 3 promoter, fragment thereof, or modified form thereof. A preferred method is by administration of an antisense oligonucleotide of human NGF promoter of exon 1 or 3. An especially preferred method is by administration of an antisense oligonucleotide to a consensus binding motif of human NGF exon 1 promoter or exon 3 promoter.

The present invention is also directed to methods for gene therapy involving altering naturally occurring transcriptional control of human NGF.

The present invention includes methods of transfecting cells and the transformed cells. Preferred embodiments of methods for transfecting cells are as follows:

1. A method of transferring a nucleic acid to a cell comprising administering to the cell a nucleic acid encoding human nerve growth factor exon 1 promoter 1-1786, 2274-2846, human nerve growth factor exon 3 promoter 1-1877, fragment thereof, or modified form thereof.

2. The method according to 1, wherein the administration is by electroporation, liposomal transfection, direct injection, vector delivery or naked deoxyribonucleic acid.

3. The method according to 2, wherein the nucleic acid comprises a consensus binding motif from human nerve growth factor exon 1 promoter selected from 1-1786, 2274-2846, human nerve growth factor exon 3 promoter 1-1877, fragment thereof, or modified form thereof.

4. The method according to 1, wherein the nucleic acid comprises deoxyribonucleic acid, ribonucleic acid, or modified form thereof.

5. The method according to 4, wherein the nucleic acid comprises a modified form of nucleic acid.

6. The method according to 5, wherein the modified form of nucleic acid comprises a phosphodiester, methylphosphonate, phosphoramidate, isopropyl phosphate triester, phosphorothioate, phosphothionate, phosphotriester or boranophosphate.

7. The method according to 1, wherein the vector delivery is by a viral vector or a modification thereof.

8. The method according to 1, wherein the vector is adenovirus, adeno-associated virus, retrovirus, herpes virus, or modifications thereof

9. The method according to claim 1, wherein the vector is an amplicon.

Embodiments of transformed cells are as followed:

1. A transformed cell comprising a nucleic acid encoding human nerve growth factor exon 1 promoter 1-1786, 2274-2846, human nerve growth factor exon 3 promoter 1-1877, fragment thereof, or modified form thereof.

2. The cell according to 1, wherein the cell comprises an animal cell.

3. The cell according to 2, wherein the cell derived from a mouse, rat, rabbit, guinea pig, hamster, pig, primate or human.

4. The cell according to 3, wherein the cell is derived from a mouse, rat, or guinea pig.

5. The cell according to 3, wherein the cell is derived from a primate or human.

6. The cell according to 5, wherein the primate is a chimpanzee, monkey or ape.

7. The cell according to 5, wherein the cell is derived from a human.

8. The cell according to 1, wherein the nucleic acid comprises nerve growth factor exon 1 promoter 1-1786, 2274-2846, fragment thereof, or modified form thereof.

9. The cell according to 8, wherein the nucleic acid comprises human nerve growth factor exon 1 promoter 1-1786, fragment thereof, or modified form thereof.

10. The cell according to 8, wherein the nucleic acid comprises human nerve growth factor exon 1 promoter to 2274-2846, fragment thereof, or modified form thereof.

11. The cell according to 1, wherein the nucleic acid is human nerve growth factor exon 3 promoter 1-1877, fragment thereof, or modified form thereof.

12. The cell according to 1, wherein the nucleic acid comprises a consensus binding motif from human nerve growth factor exon 1 promoter 1-1786, 2274-2846, human nerve growth factor exon 3 promoter 1-1877, fragment thereof, or modified form thereof.

13. The cell according to 1, wherein the cell is a yeast or bacterial cell.

14. The cell according to 12, wherein the cell is a bacterial cell.

15. The cell according to 12, wherein the cell is a yeast cell.

The present invention is also directed to methods of making animal models useful to study NGF regulation and to the resulting animals. Embodiments of such methods and resulting animals are as follows:

1. A method of transferring a nucleic acid into an animal, comprising administering to the animal a nucleic acid encoding human nerve growth factor exon 1 promoter 1-1786, 2274-2846, human nerve growth factor exon 3 promoter 1-1877, fragment thereof, or modified form thereof.

2. The method according to 1, wherein the administration is by electroporation, liposomal transfection, direct injection, vector delivery or naked deoxyribonucleic acid.

3. The method according to 2, wherein the nucleic acid comprises a consensus binding motif from human nerve growth factor exon 1 promoter 1-1786, 2274-2846, human nerve growth factor exon 3 promoter 1-1877, fragment thereof, or modified form thereof.

4. The method according to 1, wherein the nucleic acid comprises deoxyribonucleic acid, ribonucleic acid, or modified forms thereof.

5. The method according to 4, wherein the nucleic acid comprises a modified form of nucleic acid.

6. The method according to 5, wherein the modified form of nucleic acid comprises a phosphodiester, methylphosphonate, phosphoramidate, isopropyl phosphate triester, phosphorothioate, phosphothionate, phosphotriester or boranophosphate.

7. The method according to 1, wherein the vector delivery is by a viral vector or a modification thereof.

8. The method according to 1, wherein the vector is adenovirus, adenoassociated virus, retrovirus, herpes virus, or modifications thereof.

9. The method according to 1, wherein the vector is an amplicon.

10. The method according to 1, wherein the animal is a mouse, rat, rabbit, guinea pig, hamster, pig or primate.

11. The method according to 10, wherein the animal is a mouse, rat, or guinea pig.

12. The method according to 10, wherein the primate is a chimpanzee, monkey or ape.

The present invention includes animal models with human NGF exon 1 promoter or exon 3 promoter, fragment thereof, or modification thereof. Such modifications may be deletion, alteration, or inclusion of one or more consensus binding motif(s) of the endogenous NGF promoter in exon 1 and/or exon 3 of that animal which correspond to a consensus binding motif in the human NGF promoter exon 1 or exon 3. Included are animal models which are transgenic animals containing human NGF promoter of exon 1 or 3, or both exons 1 and 3, or hybrids thereof. Especially preferred animal models include animal models comprising amplicon-based NGF promoter of either exon 1 or exon 3, or both, or modifications thereof. Amplicons of the present invention differ slightly from previous examples of amplicons, where the amplicon is used to express a gene of interest. As used herein, an amplicon is a vector where the endogenous viral promoter is substituted with all or part of either human NGF promoter of exon 1 or 3, or both exons 1 and 3, or hybrids thereof, and optionally include all or part of NGF gene exons. Embodiments of the animal models of the present invention are as follows:

1 A nonhuman animal comprising human nerve growth factor exon 1 promoter 1-1786, 2274-2846, human nerve growth factor exon 3 promoter 1-1877, fragment thereof, or modified form thereof.

2. The animal according to 1, comprising a human nerve growth factor exon 1 promoter 1-1786, 2274-2846, fragment thereof, or modified form thereof.

3 The animal according to 2, wherein the nucleic acid is human nerve growth factor exon 1 promoter 1-1786, fragment thereof, or modified form thereof.

4 The animal according to 2, wherein the nucleic acid is human nerve growth factor exon 1 promoter to 2274-2846, fragment thereof, or modified form thereof.

5 The animal according to 1, wherein the nucleic acid is human nerve growth factor exon 3 promoter 1-1877, fragment thereof, or modified form thereof.

6 The animal according to 1, wherein the nucleic acid comprises a consensus binding motif from human nerve growth factor exon 1 promoter selected from 1-1786, 2274-2846 or human nerve growth factor exon 3 promoter 1-1877, or modified therefrom.

7 The animal according to 6, wherein the nucleic acid comprises a consensus binding motif is selected from the group consisting of AP1, AP2, AP3, AP4, AP5, E4TF1, CTF/NF-1, NF-KB, TFIID, TFIIIA, p53, GM-CSF or NF IL-6.

8. The animal according to 6, wherein the consensus binding motif comprises a CAAT box or TATA box.

9. The animal according to 1, wherein the nucleic acid comprises an enhancer sequence, repressor sequence or consensus binding motif for a transcription activating factor.

10 The animal according to 1, wherein the nucleic acid comprises a natural or a modified derivative of deoxyribonucleic acid or ribonucleic acid.

11. The animal according to 1, wherein the animal is transgenic.

The invention includes methods and assays for a compound capable of modifying human nerve growth factor regulation. A preferred embodiment of a method is contacting a compound with human NGF exon 1 promoter, exon 3 promoter, fragment thereof, or modification thereof. A more preferred embodiment of the present invention includes a vector comprising a modified form of human NGF exon 1 promoter or exon 3 promoter, fragment thereof, or modification thereof, such as one comprising a deletion of one or more consensus binding motif or other modification, such as a modified lariat site, altered splice donor site or splice acceptor site, or combinations thereof, cells containing such vectors comprising such vectors and assays using such cells. Embodiments of assay methods are as follows:

1. A method of identifying a compound capable of modifying human nerve growth factor regulation, comprising administering a compound to a cell, wherein the cell comprises a vector which comprises a human nerve growth factor exon 1 promoter 1-1786, 2274-2846, human nerve growth factor exon 3 promoter 1-1877, fragment thereof, or modified form thereof.

2. The method according to 1, wherein the vector comprises a human nerve growth factor exon 1 promoter 1-1786, 2274-2846, fragment thereof, or modified form thereof.

3. The method according to 2, wherein the vector comprises a human nerve growth factor exon 1 promoter selected from 1-1786, fragment thereof, or modified form thereof.

4. The method according to 2, wherein the vector comprises a human nerve growth factor exon 1 promoter selected from 2274-2846, fragment thereof, or modified form thereof.

5. The method according to 1, wherein the vector comprises a human nerve growth factor exon 3 promoter 1-1877, fragment thereof, or modified form thereof.

6. The method according to 1, wherein the vector comprises a consensus binding motif from human nerve growth factor exon 1 promoter 1-1786, 2274-2846, human nerve growth factor exon 3 promoter 1-1877, fragment thereof, or modified form thereof.

7. The method according to 1, wherein the promoter is operably linked to a gene encoding a selectable protein.

8. The method according to 7, wherein the selectable protein confers antimicrobial resistance.

9. The method according to 8, wherein the antimicrobial resistance is to neomycin, sulfonamide, penicillin, cephalosporin, aminoglycoside, tetracyclin, or modified forms thereof.

10. The method according to 1, wherein the promoter is operably linked to a gene conferring a phenotypic or genotypic modification.

11. The method according to 1, wherein the modification alters a biological pathway.

12. The method according to 10, wherein the modification confers resistance to a cytotoxin.

13 The method according to 12, wherein the cytotoxin is an exogenous compound,

14 The method according to 13, wherein the exogenous compound is an antibiotic, inorganic compound or organic compound.

15 The method according to 1, wherein the promoter is operably linked to a reporter gene.

16 The method according to 15, wherein the expression of the reporter gene is detected.

17 The method according to 16, wherein the expression is detected by fluorescence, immunological assay, enzymological assay, or modifications thereof.

18 The method according to 16, wherein the reporter gene confers detectable or selectable phenotypic change.

19 The method according to 10, wherein the reporter gene is a protein which is capable of fluorescence.

20 The method according to 19, wherein the gene is a luciferase or green fluorescent protein or modified form thereof.

21. The method according to 17, wherein the expression is detected by an immunological assay, or modification thereof.

22. The method according to 17, wherein the expression is detected by an enzymological assay, or modification thereof.

23. The method according to 22, wherein the enzymological assay is a enzyme based reporter system, or modification thereof.

24. The method according to 23, wherein the enzymological assay is based on luciferase placental alkaline phosphatase or β-galactosidase, or modifications thereof.

The present invention is also directed to a method for identifying compounds capable of modifying transcription of human NGF. Preferred embodiments of the invention are directed to a method of characterizing a compound capable of modifying initiation of transcription of human nerve growth factor exon 1 promoter or human nerve growth factor exon 3 promoter. More preferred embodiments of the invention are as follows:

1. A method for identifying a compound capable of modifying transcription of human nerve growth factor exon 1 promoter or human nerve growth factor exon 3 promoter, comprising contacting a cell comprising a nucleic acid encoding human nerve growth factor exon 1 promoter 1-1786, 2274-2846, human nerve growth factor exon 3 promoter 1-1877, fragment thereof, or modification thereof, with a compound and detecting modification of initiation of transcription.

2. The method according to 1, wherein the cell is suitable for high throughput screening.

3. The method according to 1, wherein the high throughput screening permits simultaneous evaluation of multiple compounds.

4. The method according to 1, wherein administration or detection is partially or fully automated.

5. The method according to 4, wherein administration of compound is automated.

6. The method according to 4, wherein detection is automated.

7. The method according to 1, wherein detection is based on expression of a reporter gene.

8. The method according to 7, wherein the reporter gene is luciferace, green fluorescent protein, modified form thereof, β-galactosidase, or placental alkaline phosphatase.

9. The method according to 8, wherein the reporter gene is luciferase.

10. The method according to 1, wherein the nucleic acid is in pGL3neo.

The present invention is also directed to a method for characterizing compounds capable of modifying transcription of human NGF. Preferred embodiments of the invention are directed to a method of characterizing a compound capable of modifying initiation of transcription of human nerve growth factor exon 1 promoter or human nerve growth factor exon 3 promoter. More preferred embodiments of the invention are as follows:

1. A method of characterizing a compound capable of modifying transcription of human nerve growth factor, comprising contacting a cell comprising a nucleic acid encoding human nerve growth factor exon 1 promoter 1-1786, 2274-2846, human nerve growth factor exon 3 promoter 1-1877, fragment thereof, or modification thereof, with a compound and detecting modification of transcription.

2. The method according to 1, wherein the cell is suitable for high throughput screening.

3. The method according to 1, wherein the high throughput screening permits simultaneous evaluation of multiple compounds.

4. The method according to 1, wherein administration or detection is partially or fully automated.

5. The method according to 4, wherein administration of compound is automated.

6. The method according to 4, wherein detection is automated.

7. The method according to 1, wherein detection is based on expression of a reporter gene.

8. The method according to 7, wherein the reporter gene is luciferace, green fluorescent protein, modified form thereof, β-galactosidase, or placental alkaline phosphatase.

9. The method according to 8, wherein the reporter gene is luciferase.

10. The method according to 1, wherein the nucleic acid is in pGL3neo.

11. The method according to 1, wherein a mechanism of action of the compound is determined.

12. The method according to 1, wherein a dose response relationship is determined.

The present invention is also directed to a compound capable of modifying transcription of human NGF. Preferred embodiments of the invention are directed to a compound capable of modifying initiation of transcription of human nerve growth factor exon 1 promoter or human nerve growth factor exon 3 promoter. More preferred embodiments of the invention are as follows:

1. A compound capable of binding to a human nerve growth factor exon 1 promoter 1-1786, 2274-2846, human nerve growth factor exon 3 promoter 1-1877, fragment thereof, or modification thereof.

2. The compound according to 1, wherein the compound is capable of binding to human nerve growth factor exon 1 promoter 1-1786, 2274-2846, fragment thereof, or modification thereof.

3. The compound according to 2, wherein the compound is capable of binding to human nerve growth factor exon 1 promoter 1-1786, fragment thereof, or modification thereof.

4. The compound according to claim 2, wherein the compound is capable of binding to human nerve growth factor exon 1 promoter 2274-2846, fragment thereof, or modification thereof.

5. The compound according to claim 1, wherein the compound is capable of binding to human nerve growth factor exon 3 promoter 1-1877, fragment thereof, or modification thereof.

6 A compound capable of modifying human nerve growth factor expression by directly or indirectly interacting with nucleic acid encoding human nerve growth factor exon 1 promoter 1-1786, 2274-2846, human nerve growth factor exon 3 promoter 1-1877, fragment thereof, or modification thereof.

### EXAMPLE 1

Summary of Strategy to Identify Human Nerve Growth Factor Exon 1 and Exon 3 Promoters

A brief description of the cloning strategies used to develop the cell lines described in Table 1 is provided.

DNA for the human nerve growth factor exon 3 clones was originally identified by PCR screening a human P1 genomic library (clone 0095-B8, Genome Systems). A ˜6600 bp fragment containing exon 3 was cloned into a pBS SK+ vector to yield the plasmid identified as pBSEx3. A 4329 bp fragment was isolated from the insert in pBSEx3 and subcloned into a pGL2 enhancer vector (Promega) and a pGL3 basic vector (Promega) to yield clones identified as pGL2Ex3 and pGL3Ex3, respectively. The pGL2Ex3 was transfected into mouse L929 cells and the pGL3Ex3 vector was transfected into human UC11 cells to generate the data in Table 1.

DNA for the human nerve growth factor exon 1 clones was originally identified by PCR screening a human P1 genomic library (clone 1226-G9, Genome Systems). A ˜14,000 bp fragment containing exon 1 was cloned into a pBS SK+ vector to yield the plasmid identified as pBSEx1. Two overlapping fragments were isolated from the insert in pBSEx1 and subcloned into a pGL3neo vector. The largest construct containing human nerve growth factor exon 1 is 2846 bp and is identified as pNE1KS. The second subclone from pBSEx1, identified as pNE1KE, contains the same 5′ end as pNE1KS and is truncated on the 3′ end in exon 1, resulting in an insert that is 2234 bp. pNE1KS and pNE1KE were transfected into mouse L929 cells and human UC11 cells to generate the data in Table 1.

TABLE 1
PLASMID & CELL LINE CHARACTERIZATION

| | L929 Mouse | | | UC11 Human | | |
|---|---|---|---|---|---|---|
| | Exon 3⁴ | Exon 1 KE⁵ | Exon 1 KS⁵ | Exon 3⁴ | Exon 1 KE⁶ | Exon 1 KS⁶ |
| INSERT SIZE | 4329 bp | 2234 bp | 2846 bp | 4329 bp | 2234 bp | 2846 bp |
| TRANSTECTING | pGL2 Ex3 | pNE1 KE | pNE1 KS | pGL3Ex3 | pNE1 KE | pNE1 KS |
| PLASMID | | | | | | |
| CLONING VECTOR | pGL2 | pGL3 neo | pGL3 neo | pGL3 | pGL3 neo | pGL3 neo |
| | Enhancer | | | basic | | |
| HUMAN P1 CLONE | 0095-B8 | 1226-G9 | 1226-G9 | 0095-B8 | 1226-G9 | 1226-G9 |
| COTRANSFECTION | pCDNA3 | NONE | NONE | pCDNA3 | NONE | NONE |
| VECTOR | | | | | | |
| NOVEL | 1-1877 | 1-1786 | 1-1786 | 1-1877 | 1-1786 | 1-1786 |
| SEQUENCE | | | 2274-2846 | | | 2274-2846 |
| SERUM¹ | 3.75 ± 0.34* | 1.30 ± 0.21 | 0.72 ± 0.08 | 2.48 ± 0.18* | 1.61 ± 0.13 | 1.06 ± 0.09 |
| PMA² | 1.13 ± 0.11 | 1.28 ± 0.11 | 0.57 ± 0.05 | 3.48 ± 0.18** | 2.09 ± 0.23** | 0.55 ± 0.07** |
| CALCITRIOL³ | 0.73 ± 0.07** | 0.71 ± 0.05* | 0.67 ± 0.05** | 0.98 ± 0.05 | 1.01 ± 0.23 | 0.86 ± 0.03** |

Remarks:
Fold induction over vehicle controls without serum, after serum deprivation for 48-56 hours Analyzed used Student's t test for paired samples with p < 0.05 considered significantly different from control (*p < 0.05, **p < 0.01)
¹Serum - 10% horse serum, 18 hr
²PMA - 1 μM, 18 hr
³Calcitriol - 10 nM, 18 hr
⁴Fold induction mean ± SEM from 1 cell line n = 11-17
⁵Fold induction mean ± SEM from 6 cell lines in duplicate
⁶Fold induction mean ± SEM from 6 cell lines in triplicate

Oligonucleotides and Polymerase Chain Reaction (PCR)

Oligonucleotides used to screen a genomic PI library (Genome Systems, St. Louis, Mo.) for clones containing the area of interest as well as internal oligonucleotides used in restriction digestion analysis to locate appropriately sized regions to subclone are provided in Table 2.

TABLE 2
Oligonucleotides Used in Cloning Human NGF
Promoter Regions

| | | | | Loca- |
|---|---|---|---|---|
| ID # | Species¹ | Sequence (5′-3′) | SEQUENCE | tion |
| 1 | Mouse | CTTCCTGGGCTCTAATGATGC | ID NO.1 | exon |
| | | | | 3A |
| 2 | Mouse | ATAGAAAGCTGCGTCCTTGGC | ID NO.2 | exon |
| | | | | 3B |
| 3 | Human | GGTAAAACTGTTATTGGGTCCG | ID NO.3 | exon |
| | | | | 3B |
| 4 | Human | CCAGTGGGTTTCCCTTTGACC | ID NO.4 | exon 1 |
| 5 | Human | TCTCTGCTGTGCCGGAGC | ID NO.5 | exon 1 |

¹Species indicates the species to which the sequence is homologous. Mouse oligonucleotides were found to be cross reactive to human DNA.

Primers #4 and #5 (in Table 2) were used to amplify sequence from human NGF exon 1 and primers #1 and #2 were used for exon 3 identification. Each oligonucleotide (400 nM) was used in separate reactions for exon 1 and exon 3. Template for these reactions was 1/40 the DNA from each P1 mini-prep described below. The reaction also contained 10 mM Tris-HCl pH 8.3, 50 mM KCl, 3 mM MgCl₂, 250 μM each dATP, dCTP, dGTP, dTTP, and 2.5 U Taq DNA polymerase (Perkin Elmer, Norwalk, Conn.) in 100 μl final volume with a drop of mineral oil to reduce condensation. Amplification was carried out using a Perkin Elmer 460 thermocycler programmed to 95° C. for 5 min and then cycled through 95° C., 30 s; 60° C., 30 s; 72° C., 1 min for 35 cycles. Control reactions were set up containing 500 ng of human genomic DNA as a positive template for the PCR reaction. The oligonucleotide #4 to human NGF exon 1, and #3 to exon 3B were end labeled to locate fragments containing exon 1 or 3 in blots of restriction digests and subcloned DNA.

Exon 1 Promoter Isolation

Two primers, #4 and #5 (Table 2), designed to amplify human NGF exon 1, identified three genomic clones, all of which contained exon 1. One of these clones, Clone #1226-G9, was digested with Kpn I to yield a 14 kb band which was ligated into the Kpn I site of pBS II SK+. This clone was digested with either KpnI/Eco47 III or KpnI/SmaI, and ligated into pGL3 neo to create plasmids referred to as pNE1KE, which is a truncated portion of human nerve growth factor exon 1 promoter 1 to 2234 bp insert, of the following sequence and pNE1KS which contains a 2846 bp insert of human nerve growth factor exon 1 promoter of 1 to 2846 SEQUENCE ID NO. 6 of the following sequence in Table 3.

TABLE 3
DNA SEQUENCE OF HUMAN NERVE GROWTH FACTOR EXON 1 PROMOTER

| | |
|---|---|
| GGTACCACTG CCAGCACACA GTGCCTGGCA TATGGTAGGG TCTCAATCAA | 50 |
| TAATCTTTGG AGTATTTTTG TGTTTGTTGT TTACATGTTC TTATTTACTC | 100 |
| AAGATCCTTG AAGTCCAGGG ACAGAAATAG AGGTAGTTAG GGGCAGAAAG | 150 |
| GAGCTCTTAT TAAATCAACA TGTGCAAGAA GAATATGACC AACAATTTAG | 200 |
| GGGGTGAGGA TGGAGCATAT AAGCAAACTT ATAATCTGCT TACATCACTT | 250 |
| AAAGTTTCCC CCTTACATAC CACATGGAAA AGAACCACAA GTGTCCCAAA | 300 |
| TCCTTTTGTC CTTCTGAATG ATGCCACAAG AACACATACA AATGCTCTGC | 350 |
| ATTCAACAAC CAAATTCTCT GTTATTCTAA AAGTTTAATT TCATACCCAA | 400 |
| ATTCTCAGGC AGCTATTATG TAAGGCTTGG GGCTAGTGCT TTCCAAACAA | 450 |
| GTTTATACAT GACATGATTG ATGGATGAAT TCATCCTGTT ATCTGGAAAT | 500 |
| TCTTTTGTTT AATTGACGAT GATAAATTTC CTAATGGATC ACCTCGACTA | 550 |
| TGATACTACT TTTGTAGAAA GGGCCATTCA CGGTGTTCCC TGGCCTCTTG | 600 |
| CCCTCACTTC CAAAGTGTGT TCATACACCA GCCTGTATCT GAACAAGTCA | 650 |
| GAAGTGGACA AGCCTAAGGC TGGGAAACAA CAAGGTCACA CCAAAGCTAA | 700 |
| GGCTGACTTC CAATTCCAGG GCTTTTTGCC TATTTCATCC TTCTCAGAGC | 750 |
| ATGTGTAAAT GGAATGAACT TTCTTATGGG AGCAAACGTG AAAATAGAAA | 800 |
| GAAGTAAGAC CTCAAGACTA ATCTGAATCA AGGGAGTTGG AAATGCCTAG | 850 |
| TCAGGGCTTC ATCTTGCTCA AGTGCCATCC ATTAAGGGTA AATGACCACC | 900 |
| CCCAGACTTA GGACAGGAAT CATCTGCTTC ACTAAATCCC AGTTCCCTGG | 950 |
| AGGGTGCCCT TCTGCTAAGT TGCACTGGCT GGTGTTACCA GCAATAGGGA | 1000 |
| GATTCTGTGC CCCACCTTCC CTCCCTGTTA CTCTCCTCAC ACCTACTTCT | 1050 |
| CCTCTGTGGC ATCCATACAG GGTAGGGGTC CAACCCACCT TTGCTATAGG | 1100 |
| AAGAAGCGAA GGCACAGACA AGCTCAACAC GGGAGGGAGT GGGGCTGTAA | 1150 |
| ATTTCCAAAG AGCTACGAAT CCCCTGGAAT GCTACAATTA ATGATGCACA | 1200 |
| TTTGGTGACA AATTTGACTT CAGGGGTATT TCTCCCTTGC TCATTTTATG | 1250 |
| CTGGGGTGGG AACAGCCCTG GCAGAGGGGC AGGGGAAAGT CAGGCAAGCT | 1300 |
| CTCCTGTCAG GCTGAATCGA GGGAACTCAA GAAATTTTGA AGGGTCAGGA | 1350 |
| AGAATTTGTG TGGGGCCTGG AGTGTGGAGA GGGGGGCATG GGGGCCTAGG | 1400 |
| GTTTGCTGGC TATATCAGTC TGGGGTCACA GACCCCTTGC AAAACTGATG | 1450 |
| AAAGCTGCGG ACCTTCAGCT CAGAAAAGAA TATTAGCATT GCACACAGTC | 1500 |
| GCGCAAATCA GCCTACAGTT TCAGAGGGGC CAAGGACTCC GGGAAGTTCC | 1550 |
| TGGAACCCAG GGCCTTAAGT TAAGGTCCCG GCTCTAGCTC CTGACTCCTG | 1600 |
| AAGTCCTCTG CCCCTTGTCC CCATGCTGGA CTTGCCGGGC CTGGGGGCCT | 1650 |
| TCTAGCTGGT TCTGCAGCCG CCTTCCCTTG TCAGAGGAGC TTGGGCACCT | 1700 |
| GCCCCTCGCG GAGCTCCCCC TGGGTGCTCA CCTATCCTGG GATAAGGAAA | 1750 |
| GGCGCCCCGA AGAAAAGGAG CAGCCGATGC CTGGGGCACC GAGGGCGACG | 1800 |
| CCGGGCAGAC CAGGGAGGCA CTGGCGAAGG GCAACGCGCG GGGGCAGGGC | 1850 |
| GGAGAGGTGA GGGAAGCTGC GAGCAACTCC GCCCAGCCCC AGCCAGTCGG | 1900 |
| CCCAACGACC CCTGCCGGTG CCCCAGAAAC TCCCCCTCCC GGCTTTGCGC | 1950 |
| GCGCGGCCCC TCAGACCCCA GTGGGTTTCC CTTTGACCTC TGAAGGTTTA | 2000 |
| AAGTCCTTCT CTGGCTGGGT CTGGCCAGCC CTCCAGGAGC GATCCGTCTG | 2050 |
| TAGTCCCCAG GACCCCCTCC AGCCGGGCAC CACAGCCCAG CCACAGCAGG | 2100 |
| TGCGGGGCTG GTGGTGGGGA GGGGAGGGAT GGGGGCCAGG ATTTGGAGCG | 2150 |
| TGTGACTCAG GAGTACGGGA GGAGGGGCTA AGAATTCAAG AAGCCTGTGT | 2200 |
| GAGAGCAGCT CGGCGCTCCG GCACAGCAGA GAGCGCTGGG AGCCGGAGGG | 2250 |
| GAGCGCAGCG GTGAGTCAGG CTGCCCCGAG CCGATCCCGA GAGGGGCGCA | 2300 |
| GCGCGGGCGC GGGCAGGGGT GGCTGGGCTT CGCGGGAGAG TTTGCAAGGA | 2350 |
| TACCGGTCTG GCGAGCTCTC TGGTTACCCC CGAGGCTCCC GCAGGCCGAA | 2400 |
| GAGCAGCCCG GAGAAATGTC CCGAGTGGGT GTGGGGGCGC GGGACCCTCG | 2450 |
| CGGGAGGACG AGTCGGACCG AGGGAACAGC GTTAGTTCTG GTCGTGGAGT | 2500 |
| CCCTAGTCCC AGGATGGCCT GCAGTCCAGG GAGCAGCCCT GGCGCCTGCA | 2550 |
| GAAGCCCACG GCCATGCCAG GGTCTAGCTC GAGGGCTAGA AGTGGATAAC | 2600 |
| GCGCAAGTGA GGGAGAGCGA ATGGGCGCGG AGAGGGATGC GCCGGCAGCT | 2650 |
| GGCGCGCCAG GGCGGGAGGA GTGGCGGCCA GCACCGCGGG GGGAGCGCAG | 2700 |
| AGCGCGCTGG CTGAGGTGAG CGCCGAGTAG GGAAAGTGCT GCGCGGCCCC | 2750 |
| CAGGTAGGGG GAGGAGCGGA ACGGGGCGCG CTAGACCTGG GGCAGTTCCC | 2800 |
| TCAGCGCGTC TCGGAAGGGC TGGGAGTCGT GACTGAGGGC CCCGGG | 2846 |

Sequence of human NGF insert in pGL3neo (KS). Exon 1 sequence is underlined. KE sequence ends at base 2234.

These clones were verified by restriction mapping and contain a 1787-2273 SEQUENCE ID NO. 7 sequence previously described in Cartwright M, et al., Mol Brain Res 15:67-75, 1992 and novel sequence of bases 1-1786 and 2274-2846. Novel sequence 5′ of exon 1 consists of bases 1-1786 SEQUENCE ID NO. 8, novel sequence 3′ of exon 1 consists of bases 2274-2846 SEQUENCE ID NO. 9. Exon 1 is underlined and encompasses bases 2227-2260 SEQUENCE ID NO. 10.

These clones incorporated both neomycin resistance and luciferase activity into a single vector assuring that virtually all of the transfected clones surviving in G418 media contained the exon 1 promoter region. Six cell lines from each transfection were chosen for further characterization.

Exon 3 Promoter

Exon 3 Promoter Isolation

The human P1 library was screened with cross-reactive mouse exon 3 primers, #1 and #2 (Table 2). Two clones, DMPC-HFF#1-0095-B8 and DMPC-HFF#1-0166-C12, contained exon 3. An Asp718/Pvu 1 digestion of clone #0095-B8 yielded an 6600 bp band containing exon 3. This fragment was subcloned into the Asp718 site of pBS SK+, and the resulting plasmid was referred to as pBSEx3. This clone was verified by restriction mapping and was used to generate sufficient DNA for subcloning into the luciferase expression vectors pGL2 enhancer and pGL3 basic.

Then 6600 bp from pBSEx3 DNA was digested with Hind III, which yielded a 4329 bp sequence of the NGF gene containing exon 3 and was subcloned into the pGL2 enhancer vector to create a plasmid referred to as pGL2Ex3 used for the L929 stable cell line. This clone was verified by restriction mapping and sequenced to provide the data in Table 4.

TABLE 4
DNA OF HUMAN NERVE GROWTH FACTOR EXON 3 PROMOTER

| | |
|---|---|
| AAGCTTCCCA GAAGATTCCA AGCTACAACC AAAGTTGAGA ACCACTGCTA | 50 |
| CAGAGGATTC AGGGACAGTA GAAAGGGGGA GCCAGTGAGG TAGACAGAAT | 100 |
| GTCCCACAAA TTCTGAGTGT GGAGGGATTA GGGGGATGGT GATTGACAGA | 150 |
| GTTATCAGGT TTCAATAGCT GTGGCTAAGG CCCATTAGTC CTTGAAAAAC | 200 |
| GATCAGCAGA GGCACAGTTT CCTTAAACTA TGCATTGATT GAATTTTGAA | 250 |
| CAGTTCGCCA TTAATCAAGT TTCATGGCTG AAATTGATCA AAATATTATT | 300 |
| GATTAACCTC AGGGGTCTTA AAAAGAACCC TCTCTCCTCT AGCTCTACCA | 350 |
| GGCTCGGGGT TGGTTGGACA TGGGTTCTGA GATGATAAGT CCTAGGAGTT | 400 |
| TGGTCCAGAA GAGGGAAGAA GCCCACAACA TAACTTTGGC TGTTATATGG | 450 |
| AAAGTTACAT TCAAGCAGGT GGTCTACAGC AGTGGACTGG CTCTGGGTTG | 500 |
| GCGCTTTGTC TTTGCACTGG ATACTTCACC CCATGAGGAG GAACAAGGTG | 550 |
| GAAGCCCTAA AGCAATGGTT CTTAAACTTA TGTGACTATC AGAATCACCT | 600 |
| GCAGAGCTGG TTAAACCGCA GATTGTTGTG TTTCATTCCC AGTTTCTGAT | 650 |
| TCAGTAGGTT TGTGGTAAAA CCCAAGAATT TGCATTTCTA ACATGTTCTA | 700 |
| AGATATTACT ACAATACTAC TATGGAATCA CACTTAGAGA ACCACTGCTT | 750 |
| TAAAGCATGA AACCCAGGAC AGGGCAAGCT CTAGAAGAAG TACATCAGAC | 800 |
| TTTATTAGGA TTCCTTTGTG CCCTGTAAGA AAGAATAGAA CATGATCCTT | 850 |
| AAATGAGCTG GGATTTATTT CCATGCATTT ATCAAAAGTG TGAGAGCTGA | 900 |
| TTTCTGTTTA AGTGATTACC CTATGAAAAC AGACAGGGTT TTAAAAATAG | 950 |
| ATATGCATTT GGGTTGTTTG TCCCAATGCC TTTGCATTAG AAATTTGTAA | 1000 |
| TATTTAAATT GGATTTAATT TTAGAGCCTC AACCTTCATC AGCATGAGAC | 1050 |
| TAAAAACAAT GACAACAATA TCTATAAAAA TCATTTAGAG TTTCATTATT | 1100 |
| GTGGACAGAG AATTTCTCTC TGCAGTAGTA AACTGCTTAT ATCAACACAG | 1150 |
| AATAAGACAA GGCCAAAGGC ATAGGAAATG CTGGACAGAG TTTCAAATAT | 1200 |
| AGCAATCAGA CATCCAGATG AGATTGGCAG GAGACCCTGG CCCTGGCATG | 1250 |
| CACCAAGGTG ACTTGGTCCA GAAATTGCAG ATACAGAGCC AGGGAATCTA | 1300 |
| TTGTGGTTGG CTTATAGTAG ACACCCGAAG AATGCAGATC TTCCTAGGAA | 1350 |
| TTGTGGAATT TTTTATTTAA ACCAAACTTC CCTCTTCTTC TAGTCATCCA | 1400 |
| AATTGGAGGC CATCCTAGCT TGTAGTGGAA TATCCAGAAT ATTTCCTGAG | 1450 |
| AAAGTCACTA TTACTTCTCT GGTTGCTCCA CTGATTAAAA GCGGAGGCTT | 1500 |
| TTTGTGTCCT ATAGGAAGAC GTTCAGTGGG CAGGCCCCAG AAGTGGGTAC | 1550 |
| TGCAAGTCTA TTAGCACCTC CTGATGTGTA AGGCCCATTC TATACTCCTC | 1600 |
| TCCCCTGCCC TACTCCTCTT GCAATGCATG GTGGACCTCC ACCCAGTTCT | 1650 |
| TGAACTCTGG GGCCTTTCCT TCCCTTCTTC CCTAATGAGC TCCTATTCAT | 1700 |
| CCTTAAGAAC CCTGCTCAGA TGTTACCTCC TCTATGAACA TGTCTCTAAC | 1750 |
| TAGTCTGGCC AGATAAAACC AATTTCTCCT TCCACTGTGT TTTCATATCA | 1800 |
| TGTCACATAT ACATCATACT TATCACACTG TACTTTAAAT GTTTATTTAT | 1850 |
| ATGCATGCCT TTTCCTATCT CTAGATTACT TGCTTTAGGA AGTTAAGTAT | 1900 |
| TATGTCTTAT TCTCCTTTGT GTCCCTAGCA CCTAACACTT AAAACAGTGG | 1950 |
| CCAGCACAGG ACCTGCAAGT TTAAGTGTTT AATTAATGAA ATAAATGAAT | 2000 |
| CCCAATTTTG GGATGAGAGA AAGCACTACT TAAGCATCTA GTAGCAATGC | 2050 |
| AGCCTGGAAA ACATTCAAAG TCACGGAATC TCAGATGATC AGAGCCAAAG | 2100 |
| GGGACCTTAG CTGTCATCTG TGCCAGCTTC TTATCCTATA GAGGAGAAAG | 2150 |
| CTCAAAGATG AAATGAATCT CCTTCTATAC AGGAGAAGCT CAGAGTGAAC | 2200 |
| TGAATCAGAA TGCGGGTGTG TGGGTTCCAG CCTGCAACCT TTCAGGTTTA | 2250 |
| GCCAAACACC CAGATGAAGG GTTTATGGAC TAGACGAAAC CATCTTCCCA | 2300 |
| TGAGTAATGG GACCAGATAA TGCCCACCTC TTACCCTGGG GACACGCCAT | 2350 |
| TCTCCCTCTC CCATGGTAAC TCCAACCCTG GGAGAGCATG AAAATGTTCT | 2400 |
| TTGTCACAGA ATGTAACCTT TTAAAGAGTG TCTGAGTATG CATTTTCATC | 2450 |
| ACTAGCCTTC AACCCCAATT GAGTATTGAA AGGTTTTTCT GGTACTTTCT | 2500 |
| GGAGCAAGAA GACTATTTTG AGCAAGATGG GAAAGGAAGA AGAATGGAGA | 2550 |
| CATCCCAGGG CTTAATTTCA TGATTTCTAG TAACTTGAAG ATCACTTTAG | 2600 |
| AGGTCCTTGC TACCTCCCCA TTCTCCAACT CCTCTTCGTG GTTGGAATTT | 2650 |
| GGGGAGCGAT GGTGGGTTTT CTGACATTTG CTTTCATAGC ACAAGCTGAG | 2700 |
| AGGGAGTTGG ATGAAGATAT GTGGTGGGGA TCCACGCTGG AAAAAGATAT | 2750 |
| CACAGGGAGA AGATTTTTTT GAAGTTGAAG AGAGAATACG GACAGGAAAG | 2800 |
| TTAAGATGTC ATTGTAGAAC TTTATTGGGA GGGCATCTCC ACCCTACAAC | 2850 |
| AAATTCTGTG ATGGACATAA TCATTCATTC ATTTATCCGT AAATATCACC | 2900 |
| CTCTTGTTCA AAGCCCTCCA CTGCCTTCCT AATATCCTGA GGATAAAACC | 2950 |
| ATAGCTCCTT GCTGTGTCTC TGTAGACCTG GCTCTTCCTG GCTCTCCAGC | 3000 |
| TCATTTTCTA GGTCTCGTTA CTTCATGCTC AGAACCTTTG TCTTGTTTCT | 3050 |
| AGCTCAGGGC CTTTGCACTT GTTCTTGCTG CCTAGAATGT TCTCTCGCTC | 3100 |
| ATTCCTTCTC ATCCTCCAGA TCTCAACTTG AAGGCCATCT CCTCAGAGCT | 3150 |
| CCTCGCTGAG CGTCCTGTCT ACAGTGGCCC CTCGATACAT CCTGCAGTTG | 3200 |
| CTCTCTATCA TCAGACCCTG TAATTGCCTT CATGGCATAT AAAGAATCTG | 3250 |
| GAGATATCTT GCTTATTTAC ACAACACTGT AAGCTCCATG AGAGCAGAGG | 3300 |
| CCTTGTTTGT CTTGTTTACT GCTGCTCAGC ACCAAAAACA GTGCCTGGCA | 3350 |
| CATAGTCGGT GCCCAGAAAA TATTGTGAAT GAATGAAGTG CCTACATAGA | 3400 |
| TTACATTATA GAAGTGAGAG GAGAATAGAA AACTTCCATT GTTTCTAGAA | 3450 |
| ACTACAGCCT AAAATTGATT TTTTAAAATT GTATCAGCTC CATAGCTTCC | 3500 |
| AATCCTAAAA TCTGCCTTTC AGTGTGGTAC TCTGAGATTC CTGTCTGATT | 3550 |
| CTGTGAGAGC TCCACATTCT CTCTCAAATG GTCAGTCTGT CTTATTTGTC | 3600 |
| ACCATTACTG ATCTGCATTT TTATCAAAGC ACCAACTTGC TCTGAATTGT | 3650 |
| CAGGGATTTT GCGTCTGTAT AAGGTATTTT AGGCTGGTTC AGAGTTGGAT | 3700 |
| GTGTTATGTC TGCATGTGTA ATGTACTGAA CAATTTCTAT TTTGATGCCA | 3750 |
| GATTAGGGAT CTGCTGGGGC AAGACTTTGG CATGTGTCTA GAAACACCTG | 3800 |
| CACTAGGTGC AAGATCAGCC ATGGACTGTG TCCAGGCTGA AACCAAAAGG | 3850 |
| TATGGCGCAA GAGTGAGAGG CAGGTGCCAC CACAGGACCA TGAGAGGCCA | 3900 |
| AGCTCCGGTA AATTTTGGTA GACCAAATTC TAGCTCCTTC CTGGGCCTTG | 3950 |
| ATGCTGGTAA AATCCCAGAA CTCAAGGAAA TGGAATTTGT CCTATTGGCA | 4000 |
| CATGCCTCCC CACTGTGTAG GGCACAGGGA ATGTGGTGAG GTACAGTCTA | 4050 |
| ATGCCAGCTC TCCCCCTCCA CAGAGTTTTG GCCAGTGGTC GTGCAGTCCA | 4100 |
| AGGGGCTGGA TGGCATGCTG GACCCAAGCT CAGCTCAGCG TCCGGACCCA | 4150 |
| ATAACAGTTT TACCAAGGGA GCAGCTTTCT ATCCTGGCCA CACTGAGGTA | 4200 |
| AGTGCCTAAG GGACCTTGGC CTTGCCAAGG TCCTCCCTCT GCAGCTGCCA | 4250 |
| GAAGCAGGAG TCCCAAGTGA CAGGACCTGA GAGGGCAAGT CAGAACCAAC | 4300 |
| TGCTGAGCAG CAGGGGCCTA GAGAAGCTT | 4329 |

Sequence of human NGF gene insert in Hind III site of pG2 enhancer. Exon 3 sequence is underlined.

Entire sequence of the pGL2Ex3 plasmid insert is shown above SEQUENCE ID NO. 11 with the novel sequence comprised of bases 1-1877 SEQUENCE ID NO. 12. Base 1877 is equivalent to base number 1 as previously reported by Ullrich et al (accession number VO1511). Exon 3B sequence is underlined and encompasses bases 4074-4197 SEQUENCE ID NO. 13. The pGL2Ex3 plasmid was digested with Hind III and the same insert subcloned into the Hind III site of pGL3 basic vector to yield the plasmid referred to as pGL3Ex3 used for the UC11 stable cell line.

Stable transfectants of UC11 or L929 cells containing the pGL3Ex3 plasmid or the pGL2Ex3 plasmid and the G418 resistant plasmid pcDNA3, were selected on the basis of their ability to survive in media containing 600 μg/ml G418 and express luciferase activity. From these co-transfections, 34% and 36% of clones screened showed luciferase activity in L929 and UC11 cells, respectively, indicating incorporation of the exon 3 promoter region. One cell line from each transfection was selected for further evaluation and a number of assays were conducted to characterize the cell lines and test functionality of the NGF promoter region in these cells.

A luciferase-based reporter plasmid was used to investigate the nerve growth factor exon 1 and exon 3 promoters. The thymidine kinase promoter and neomycin resistance gene, excised from pMC1neo (Stratagene, LaJolla, Calif.) using Xho I, were cloned into the Sal I cut plasmid pGL3-basic (Promega, Madison, Wis.). The resulting vector was designated “pGL3-neo” and is 5960 bp. One advantage of this vector is the dual incorporation of a selectable marker, here, neomycin resistance, and a reporter gene, here the luciferase gene. This vector avoids the necessity of co-transfection, and is stable over multiple passages and the transfected cell line maintains a high level of desired protein expression, here luciferase. Thus, this vector is particularly desirable for high-throughput assays. Another advantage is the small size, which permits relatively large insertions of the promoter or other control elements of interest. Still another advantage of this vector is that incorporation of the selectable gene and promoter, here tk-neo, affects only one of the otherwise unique restriction sites, Mlu 1, in the pGL3-basic vector. Thus, the remaining unique restriction endonuclease sites, Kpn 1, Sac I, Nhe I, Sma I, Xho I, Bgl II, and Hind III, are unaffected. Other vectors, using SV40 promoter or RSV promoter, instead of the thymidine kinase promoter, were tested. The complete sequence of pGL3-neo SEQUENCE ID NO. 14 is provided in Table 5:

TABLE 5
Sequence of pGL3-neo

GGTACCGAGCTCTTACGCGTGCTAGCCCGGGCTCGAGATCTGCGATCTAAGTAAGCTTGGCATTCCG
GTACTGTTGGTAAAGCCACCATGGAAGACGCCAAAAACATAAAGAAAGGCCCGGCGCCATTCTATC
CGCTGGAAGATGGAACCGCTGGAGAGCAACTGCATAAGGCTATGAAGAGATACGCCCTGGTTCCTG
GAACAATTGCTTTTACAGATGCACATATCGAGGTGGACATCACTTACGCTGAGTACTTCGAAATGTC
CGTTCGGTTGGCAGAAGCTATGAAACGATATGGGCTGAATACAAATCACAGAATCGTCGTATGCAG
TGAAAACTCTCTTCAATTCTTTATGCCGGTGTTGGGCGCGTTATTTATCGGAGTTGCAGTTGCGCCC
GCGAACGACATTTATAATGAACGTGAATTGCTCAACAGTATGGGCATTTCGCAGCCTACCGTGGTGT
TCGTTTCCAAAAAGGGGTTGCAAAAAATTTTGAACGTGCAAAAAAAGCTCCCAATCATCCAAAAAA
TTATTATCATGGATTCTAAAACGGATTACCAGGGATTTCAGTCGATGTACACGTTCGTCACATCTCA
TCTACCTCCCGGTTTTAATGAATACGATTTTGTGCCAGAGTCCTTCGATAGGGACAAGACAATTGCA
CTGATCATGAACTCCTCTGGATCTACTGGTCTGCCTAAAGGTGTCGCTCTGCCTCATAGAACTGCCT
GCGTGAGATTCTCGCATGCCAGAGATCCTATTTTTGGCAATCAAATCATTCCGGATACTGCGATTTT
AAGTGTTGTTCCATTCCATCACGGTTTTGGAATGTTTACTACACTCGGATATTTGATATGTGGATTTC
GAGTCGTCTTAATGTATAGATTTGAAGAAGAGCTGTTTCTGAGGAGCCTTCAGGATTAGAAGATTCA
AAGTGCGCTGCTGGTGCCAACCCTATTCTCCTTCTTCGCCAAAAGCACTCTGATTGACAAATACGAT
TTATCTAATTTACACGAAATTGCTTCTGGTGGCGCTCCCCTCTCTAAGGAAGTCGGGGAAGCGGTTG
CCAAGAGGTTCCATCTGCCAGGTATCAGGCAAGGATATGGGCTCACTGAGACTACATCAGCTATTCT
GATTACACCCGAGGGGGATGATAAACCGGGCGCGGTCGGTAAAGTTGTTCCATTTTTTGAAGCGAA
GGTTGTGGATCTGGATACCGGGAAAACGCTGGGCGTTAATCAAAGAGGCGAACTGTGTGTGAGAGG
TCCTATGATTATGTCCGGTTATGTAAACAATCCGGAAGCGACCAACGCCTTGATTGACAAGGATGG
ATGGCTACATTCTGGAGACATAGCTTACTGGGACGAAGACGAACACTTCTTCATCGTTGACCGGCTG
AAGTCTCTGATTAAGTACAAAGGCTATCAGGTGGCTCCCGCTGAATTGGAATCCATCTTGCTCCAAC
ACCCCAACATCTTCGACGCAGGTGTCGCAGGTCTTCCCGACGATGACGCCGGTGAACTTCCCGCCGC
CGTTGTTGTTTTGGAGCACGGAAAGACGATGACGGAAAAAGAGATCGTGGATTACGTCGCCAGTCA
AGTAACAACCGCGAAAAAGTTGCGCGGAGGAGTTGTGTTTGTGGACGAAGTACCGAAAGGTCTTAC
CGGAAAACTCGACGCAAGAAAAATCAGAGAGATCCTCATAAAGGCCAAGAAGGGCGGAAAGATCG
CCGTGTAATTCTAGAGTCGGGGCGGCCGGCCGCTTCGAGCAGACATGATAAGATACATTGATGAGT
TTGGACAAACCACAACTAGAATGCAGTGAAAAAAATGCTTTATTTGTGAAATTTGTGATGCTATTGC
TTTATTTGTAACCATTATAAGCTGCAATAAACAAGTTAACAACAACAATTGCATTCATTTTATGTTTC
AGGTTCAGGGGGAGGTGTGGGAGGTTTTTTAAAGCAAGTAAAACCTCTACAAATGTGGTAAAATCG
ATAAGGATCCGGCAGTGTGGTTTTGCAAGAGGAAGCAAAAAGCCTCTCCACCCAGGCCTGGAATGT
TTCCACCCAATGTCGAGCAGTGTGGTTTTGCAAGAGGAAGCAAAAAGCCTCTCCACCCAGGCCTGG
AATGTTTCCACCCAATGTCGAGCAAACCCCGCCCAGCGTCTTGTCATTGGCGAATTCGAACACGCAG
ATGCAGTCGGGGCGGCGCGGTCCCAGGTCCACTTCGCATATTAAGGTGACGCGTGTGGCCTCGAAC
ACCGAGCGACCCTGCAGCCAATATGGGATCGGCCATTGAACAAGATGGATTGCACGCAGGTTCTCC
GGCCGCTTGGGTGGAGAGGCTATTCGGCTATGACTGGGCACAACAGACAATCGGCTGCTCTGATGC
CGCCGTGTTCCGGCTGTCAGCGCAGGGGCGCCCGGTTCTTTTTGTCAAGACCGACCTGTCCGGTGCC
CTGAATGAACTGCAGGACGAGGCAGCGCGGCTATCGTGGCTGGCCACGACGGGCGTTCCTTGCGCA
GCTGTGCTCGACGTTGTCACTGAAGCGGGAAGGGACTGGCTGCTATTGGGCGAAGTGCCGGGGCAG
GATCTCCTGTCATCTCACCTTGCTCCTGCCGAGAAAGTATCCATCATGGCTGATGCAATGCGGCGGC
TGCATACGCTTGATCCGGCTACCTGCCCATTCGACCACCAAGCGAAACATCGCATCGAGCGAGCAC
GTACTCGGATGGAAGCCGGTCTTGTCGATCAGGATGATCTGGACGAAGAGCATCAGGGGCTCGCGC
CAGCCGAACTGTTCGCCAGGCTCAAGGCGCGCATGCCCGACGGCGAGGATCTCGTCGTGACCCATG
GCGATGCCTGCTTGCCGAATATCATGGTGGAAAATGGCCGCTTTTCTGGATTCATCGACTGTGGCCG
GCTGGGTGTGGCGGACCGCTATCAGGACATAGCGTTGGCTACCCGTGATATTGCTGAAGAGCTTGG
CGGCGAATGGGCTGACCGCTTCCTCGTGCTTTACGGTATCGCCGCTCCCGATTCGCAGCGCATCGCC
TTCTATCGCCTTCTTGACGAGTTCTTGTGAGGGGATCGGCAATAAAAAGACAGAATAAAACGCACG
GGTGTTGGGTCGTTTGTTCGGATCCGTCGACCGATGCCCTTGAGAGCCTTCAACCCAGTCAGCTCCT
TCCGGTGGGCGCGGGGCATGACTATCGTCGCCGCACTTATGACTGTCTTCTTTATCATGCAACTCGT
AGGACAGGTGCCGGCAGCGCTCTTCCGCTTCCTCGCTCACTGACTCGCTGCGCTCGGTCGTTCGGCT
GCGGCGAGCGGTATCAGCTCAGTCAAAGGCGGTAATACGGTTATCCACAGAATCAGGGGATAACGC
AGGAAAGAACATGTGAGCAAAAGGCCAGCAAAAGGCCAGGAACCGTAAAAAGGCCGCGTTGCTGG
CGTTTTTCCATAGGCTCCGCCCCCCTGACGAGCATCACAAAAATCGACGCTCAAGTCAGAGGTGGCG
AAACCCGACAGGACTATAAAGATACCAGGCGTTTCCCCCTGGAAGCTCCCTCGTGCGCTCTCCTGTT
CCGACCCTGCCGCTTACCGGATACCTGTCCGCCTTTCTCCCTTCGGGAAGCGTGGCGCTTTCTCAAT
GCTCACGCTGTAGGTATCTCAGTTCGGTGTAGGTCGTTGGCTCCAAGCTGGGCTGTGTGCACGAACC
CCCCGTTCAGCCCGACCGCTGCGCCTTATCCGGTAACTATCGTCTTGAGTCCAACCCGGTAAGACAC
GACTTATCGCCACTGGCAGCAGCCACTGGTAACAGGATTAGCAGAGCGAGGTATGTAGGCGGTGCT
ACAGAGTTCTTGAAGTGGTGGCCTAACTACGGCTACACTAGAAGGACAGTATTTGGTATCTGCGCTC
TGCTGAAGCCAGTTACCTTCGGAAAAAGAGTTGGTAGCTCTTGATCCGGCAAACAAACCACCGCTG
GTAGCGGTGGTTTTTTTGTTTGCAAGCAGCAGATTACGCGCAGAAAAAAAGGATCTCAAGAAGATC
CTTTGATCTTTTCTACGGGGTCTGACGCTCAGTGGAACGAAAACTCACGTTAAGGGATTTTGGTCAT
GAGATTATCAAAAAGGATCTTCACCTAGATCCTTTTAAATTAAAAATGAAGTTTTAAATCAATCTAA
AGTATATATGAGTAAACTTGGTCTGACAGTTACCAATGCTTAATCAGTGAGGCACCTATCTCAGCGA
TCTGTCTATTTCGTTCATCCATAGTTGCCTGACTCCCCGTCGTGTAGATAACTACGATACGGGAGGG
CTTACCATCTGGCCCCAGTGCTGCAATGATACCGCGAGACCCACGCTCACCGGCTCCAGATTTATCA
GCAATAAACCAGCCAGCCGGAAGGGCCGAGCGCAGAAGTGGTCCTGCAACTTTATCCGCCTCCATC
CAGTCTATTAATTGTTGCCGGGAAGCTAGAGTAAGTAGTTCGCCAGTTAATAGTTTGCGCAACGTTG
TTGCCATTGCTACAGGCATCGTGGTGTCACGCTCGTCGTTTGGTATGGCTTCATTCAGCTCCGGTTC
CCAACGATCAAGGCGAGTTACATGATCCCCCATGTTGTGCAAAAAAGCGGTTAGCTCCTTCGGTCCT
CCGATCGTTGTCAGAAGTAAGTTGGCCGCAGTGTTATCACTCATGGTTATGGCAGCACTGCATAATT
CTCTTACTGTCATGCCATCCGTAAGATGCTTTTCTGTGACTGGTGAGTACTCAACCAAGTCATTCTG
AGAATAGTGTATGCGGCGACCGAGTTGCTCTTGCCCGGCGTCAATACGGGATAATACCGCGCCACA
TAGCAGAACTTTAAAAGTGCTCATCATTGGAAAACGTTCTTCGGGGCGAAAACTCTCAAGGATCTTA
CCGCTGTTGAGATCCAGTTCGATGTAACCCACTCGTGCACCCAACTGATCTTCAGCATCTTTTACTTT
CACCAGCGTTTCTGGGTGAGCAAAAACAGGAAGGCAAAATGCCGCAAAAAAGGGAATAAGGGCGA
CACGGAAATGTTGAATACTCATACTCTTCCTTTTTCAATATTATTGAAGCATTTATCAGGGTTATTGT
CTCATGAGCGGATACATATTTGAATGTATTTAGAAAAATAAACAAATAGGGGTTCCGCGCACATTTC
CCCGAAAAGTGCCACCTGACGCGCCCTGTAGCGGCGCATTAAGCGCGGCGGGTGTGGTGGTTACGC
GCAGCGTGACCGCTACACTTGCCAGCGCCCTAGCGCCCGCTCCTTTCGCTTTCTTCCCTTCCTTTCTC
GCCACGTTCGCCGGCTTTCCCCGTCAAGCTCTAAATCGGGGGCTCCCTTTAGGGTTCCGATTTAGTG
CTTTACGGCACCTCGACCCCAAAAAACTTGATTAGGGTGATGGTTCACGTAGTGGGCCATCGCCCTG
ATAGACGGTTTTTCGCCCTTTGACGTTGGAGTCCACGTTCTTTAATAGTGGACTCTTGTTCCAAACTG
GAACAACACTCAACCCTATCTCGGTCTATTCTTTTGATTTATAAGGGATTTTGCCGATTTCGGCCTAT
TGGTTAAAAAATGAGCTGATTTAACAAAAATTTAACGCGAATTTTAACAAAATATTAACGTTTACAA
TTTCCCATTCGCCATTCAGGCTGCGCAACTGTTGGGAAGGGCGATCGGTGCGGGCCTCTTCGCTATT
ACGCCAGCCCAAGCTACCATGATAAGTAAGTAATATTAAGGTACGGGAGGTACTTGGAGCGGCCGC
AATAAAATATCTTTATTTTCATTACATCTGTGTGTTGGTTTTTTGTGTGAATCGATAGTACTAACATA
CGCTCTCCATCAAAACAAAACGAAACAAAACAAACTAGCAAAATAGGCTGTCCCCAGTGCAAGTGC
AGGTGCCAGAACATTTCTCTATCGATA

### EXAMPLE 2

Protocol to Amplify P₁ Genomic DNA

Glycerol stocks of bacterial cells containing P₁ genomic DNA (Genome Systems) were used to inoculate Luria Broth (LB) containing 25 μg/ml kanamycin. The cultures were grown overnight at 37° C. and mini preps prepared by a modified alkaline lysis method as recommended by the manufacturer. DNA was used within 24 hours for restriction analysis or stored in small aliquots at −20° C. to avoid repeated thawing and freezing. For DNA subcloning, 20 mls of overnight culture were processed as 1.5 ml aliquots, pooled, digested with the appropriate restriction enzymes and size fractionated on a gel.

### EXAMPLE 3

Subcloning of P₁ Fragments

To isolate DNA for restriction digestion analysis and locate an appropriately sized piece for subcloning, the P₁ DNA was size fractionated by agarose gel electrophoresis and the gel was soaked in 0.2 N HCl for 10 min, rinsed in distilled H₂O, denatured in 0.5 N NaOH/1.5 M NaCl 2 times, 15 minutes each and neutralized in 1.5 M NaCl/1M Tris-HCl (pH 7.4) 2 times, 15 minutes each. DNA was transferred onto Nytran membranes (Schleicher & Schuell, Keene, N.H.) by downward capillary action for 1-3 hours. When an appropriate fragment was identified by hybridization, a duplicate FIGE gel was run and the band excised from the agarose gel and purified for ligation using Geneclean (Bio 101, La Jolla, Calif.).

Labeling Oligonucleotides for Probes

End-labeling of oligonucleotides as probes for exon 3, was performed using γ[³²P] ATP (Amersham, Arlington Heights, Ill.), specific activity >5000 Ci/mmol, in a 2:1 pmol ratio with oligonucleotide. The oligonucleotide was denatured by placing in boiling water for 2 minutes, then mixed with the radioactive ATP and dried in a vacuum desiccator. The mix was resuspended in 50 mM Tris-HCl (pH 7.6), 10 mM MgCl₂, 5 mM DTT, 10 units T4 polynucleotide kinase (PNK) (Gibco/BRL, Gaithersburg, Md.) and the reaction incubated at 37° C. After 1 hour another 10 units T4 PNK was added and the reaction continued another hour. The unincorporated ATP was removed with a select-D, G-25 column (5 Prime-3 Prime, West Chester, Pa.) according to manufacturers instructions. Non-radioactive exon 1 oligonucleotide probes were labeled using ECL 3′ oligolabeling protocol recommended by the manufacturer (Amersham).

Hybridization Conditions

Hybridization of exon 3 blots was carried out by first pre-hybridizing blots in 6×SSC, 5× Denhart's, 100 μg/ml salmon sperm DNA, 0.5% SDS, 0.2 M NaPO₄ (pH 7.0), at 50° C. for 3-6 hours. Fresh hybridization solution identical to pre-hybridization solution but including 10% dextran sulfate and ˜10 ng/ml end labeled oligonucleotide was incubated with the blots at 50° C. for 15-18 hours. The blots were washed with 6×SSC/0.5% SDS at 52° C. 2 times quickly, then 2 times 15 min each. Wash solution was replaced with 2×SSC/0.5% SDS, and blots washed for 15 min more at 52° C. Hybridization and washing of the exon 1 blots was done as recommended by the manufacturer with the more stringent wash being completed at 45° C. To detect the signal, radioactive blots were placed on a phosphorimager screen for 5-24 hours and scanned by a Molecular Dynamics SF phosphorimager using ImageQuant software analysis (Molecular Dynamics, Sunnyvale, Calif.). ECL screened blots were placed on film (Hyperfilm ECL, Amersham) for 10 to 30 minutes.

Ligation and Transformation Conditions

Vector DNA (5 μg, pBS SK+ (Stratagene, La Jolla, Calif.), pGL2 Enhancer, pGL3 basic (Promega, Madison, Wis.), or pGL3 neo) was digested with the appropriate restriction endonuclease and incubated with 25-50 units calf intestinal alkaline phosphatase (Gibco/BRL, Gaithersburg, Md.) to remove the 5′ phosphate group and reduce self-ligation. The reaction was carried out in 50 mM Tris-HCl (pH 8.5), 0.1 mM EDTA at 37° C. for 30 minutes. The DNA was run on a 1% agarose gel (Ultrapure agarose, Gibco/BRL) at 80-100 volts, and the linearized band excised and purified with Geneclean. Both insert and vector DNA were diluted to ˜50 ng/μl and ligated in a 3:1 ratio for 15-18 hours at 14° C. in 50 mM Tris-HCl (pH 7.6), 10 mM MgCl₂, 1 mM ATP, 1 mM DTT, 5% polyethylene glycol-8000 with 0.5 units T4 ligase.

Transformation was carried out by mixing 50 μl of maximum efficiency DH5α cells (Gibco/BRL) with 2 μl of undiluted ligation reaction mix on ice for 30 minutes. The cells were heat shocked 40 sec at 42° C., returned to ice for 2 min and 950 μl SOC media was added to begin recovery. The cells were shaken at 225 rpm in SOC at 37° C. for 1 hour and 200 μl of this suspension was spread on an agar plate containing 50 μg/ml ampicillin. Agar plates were incubated at 37° C. overnight for growth of colonies. Clones containing the appropriate plasmid insert were identified by restriction analysis and confirmed by sequencing.

### EXAMPLE 5

Cell Culture

(All cell culture reagents were from Gibco/BRL (Gaithersburg, Md.) unless otherwise noted.)

L929 mouse fibroblast cells (ATCC, Rockville, Md.) were grown in Dulbecco's modified Eagle's medium (DMEM) containing 10% horse serum, penicillin (50 μg/ml), streptomycin (50 μg/ml), neomycin (100 μg/ml), and glutamine (1 mM). Cells were maintained at 37° C. in 5% CO₂, fed every 3-4 days and passaged once per week. When serum free media was used before luciferase assays, it contained DMEM:Ham's F12 (3:1), insulin (5 μg/ml), transferring (5 μg/ml), sodium selenite (5 ng/ml), penicillin (50 μg/ml), streptomycin (50 μg/ml), neomycin (100 μg/ml) and glutamine (1 mM).

UC11 human astrocytoma cells (Liwnicz, et. al. 1986) were grown in RPMI 1640 containing 10% fetal bovine serum, 20 mM HEPES, penicillin (50 μg/ml), streptomycin (50 μg/ml), neomycin (100 μg/ml), and glutamine (1 mM). Cells were maintained at 37° C. in 5% CO₂, fed every 3-4 days and passaged once per week. When serum free media was used before luciferase assays, it contained RPMI:Ham's F12 (3:1), 20 mM HEPES, insulin (5 μg/ml), transferring (5 μg/ml), sodium selenite (5 ng/ml), penicillin (50 μg/ml), streptomycin (50 μg/ml), neomycin (100 μg/ml) and glutamine (1 mM).

Since geneticin (G418) resistance would be used as a selection tool, a G418 concentration curve was done and it was determined that 600 μg/ml was the minimum concentration G418 necessary to kill all the wild type cells in 13 days.

### EXAMPLE 6

Stable Transfections

Exon 1 clones were prepared by electroporation of 10 μg pNE1KE or pNE1KS DNA into 5×10⁶ L929 or UC11 cells. The exon 3 clones required co-transfection with pcDNA3 (Invitrogen, San Diego, Calif.) containing the neomycin resistance gene which confers G418 resistance allowing selection of transfectants. For exon 3 clones, L929 cells were electroporated with 10 μg pGL2Ex3 DNA and 1 μg pcDNA3 and UC11 cells were electroporated with 10 μg pGL3Ex3 DNA and 1 μg pcDNA3. All plasmids were linearized with Xho I prior to electroporation according to the procedure outlined below.

On day 1 electroporation was carried out by placing cells and DNA in 1 ml Hank's Balanced Salt Solution (HBSS) and pre-incubating on ice for 5 min. Current was applied at room temperature at 750 V for 9 msec. Cells remained in the chamber for a 2 minute recovery phase, were resuspended in normal L929 or UC11 media, and plated in a 100 mm dish.

On day 3, cells were split 1:10 with trypsin and replated into 100 mm dishes in media containing 400 μg/ml G418. The concentration of G418 was increased by feeding cells every other day with media containing 600 μg/ml G418, 800 μg/ml G418, and back to 600 μg/ml G418. Media containing 600 μg/ml G418 was then replaced every 3-4 days until individual colonies of cells could be seen and harvested. Cells were harvested by removing media from plate, and scraping the cells from the dish using a drop of trypsin and a pipette tip.

### EXAMPLE 7

Luciferase Assay

Cells were plated at 5,000 cells/well in 96 well dishes in serum containing media described above. The next day cells were washed twice and incubated for an additional 48-56 hours in serum free media. Cells were treated with 1 μM PMA, 10 nM calcitriol or 10% horse serum and luciferase activity was determined 18 hours later using a Promega kit (catalog #E1500). Briefly, media was aspirated and cells were lysed in 200 μl cell lysis buffer (containing 25 mM Tris-phosphate, pH 7.8, 2 mM DTT, 2 mM 1,2-diaminocyclohexane-N,N,N′N′-tetraacetic acid, 10% glycerol, 1% triton X-100). 100 μl cell/buffer solution was transferred to a white Dynatech microlite 2, 96 well dish. Luciferase activity was detected in a MicroLumat LB 96 P luminometer (Wallac Inc, Gaithersburg, Md.) for 10 seconds following automatic injection of 100 μl 470 μM luciferin.

### EXAMPLE 8

Consensus binding motifs in the sequences human nerve growth factor exon 1 and exon 3 promoters were determined using MacVector, Ver 4.0, (IBI, Inc, NewHaven, Conn.). Putative consensus sequences were scanned for relatively high fidelity to the consensus binding motif and are preferred consensus binding motifs in human nerve growth factor exon 1 and exon 3 promoters. Table 6 provides a partial list of consensus binding motifs.

TABLE 6
CONSENSUS BINDING MOTIFS IN HUMAN NERVE GROWTH FACTOR EXON 1 AND 3 PROMOTERS

| | | Beginning of Con- | Beginning of | | |
|---|---|---|---|---|---|
| | | sensus binding motif | Consensus binding | | |
| | | in NGF exon 1 | motif in NGF exon 3 | | |
| | Consensus binding | promoter Note: +/− | promoter | | |
| | motif Note: consensus | indicates location on | Note: + −indicates | | |
| | sequence in paren- | positive or negative | location on positive | Literature | |
| Name | theses | strand | or negative strand | Reference | |
| E2F | (TTTCGCGC) | +1945 | | EMBO J 6:2061, | |
| | SEQUENCE ID NO. 15 | | | 1987 | |
| AP1 | (TKASTMA) | +95,−192,−821,+824, | +185,+261,−306,−589 | Cell 49:741, | |
| | SEQUENCE ID NO. 16 | −830,847,−1212,−1598, | +647,−653,−1053, | 1987 | |
| immediate early | | +2153,−2159,+2262, | +1390,−1488,+2201, | | |
| gene response | | −2268,2836 | −2207,−2307,+2400, | | |
| element | | | +3595,+3605,+4328, | | |
| | | | −267,+300,−1396, | | |
| | | | +1482,+2301,−3611 | | |
| AP2 | (CCSCRGGC) | −1646,−1786,+2389 | | Nuc Acids Res | |
| | SEQUENCE ID NO. 17 | | | 20:3,1992 | |
| AP3 | (TGTGGWWW) | −274,+1370 | +661,+1352,+116, | Nuc Acids Res | |
| protein kinase SEQUENCE ID NO. 18 | | −1608,+2472 | 20:3, 1992; | | |
| C responsive | | | | Nature 329:648, | |
| element | | | | 1987 | |
| AP4 | (CAGCTGTGG) | −293,+649,−699, | −37,−50,+166,−171, | Genes Dev | |
| | SEQUENCE ID NO. 19 | +1051,+1263,+1452, | +204,+477,−482, | 2:267, 1988 | |
| protein kinase | | −2088,−2099,+2097, | −609,−750,+1583, | | |
| C responsive | −2229,+2256,+2314, | −2113,+2115,−2957, | | | |
| element | | +2534,+2646,−2651 | −3948,+3761,+3816, | | |
| | | | −4247,−4305,+4308 | | |
| AP5 | (CTGTGGAATG) | −275,−359,+1054, | +480,+720,+1351, | EMBO J 8:1455, | |
| | SEQUENCE ID NO. 20 | +1172,−1496 | −1787,+3714,+3775, | 1989 | |
| immediate early | | | −4073 | | |
| gene response | | | | | |
| element | | | | | |
| APRT | (GCCCCACC) | +1009 | | Mol Cell Biol | |
| | SEQUENCE ID NO. 21 | | | 8:2536,1988 | |
| ISRE | (GGGAAATAGAAAST) | +789,+1823,−1987 | +3240,−3745,+3974 | Genes Dev | |
| | SEQUENCE ID NO. 22 | | | 2:383,1988 | |
| interferon stimulated | | | | | |
| response element | | | | | |
| E2aE | (TGGGAATT) | −407,+494,−941,−1157 | −641,+859,+1345, | Nucleic-Acids- | |
| adenovirus | SEQUENCE ID NO. 23 | | −2326,+2642,−3967 | Res. 1991 Dec | |
| promoter element | | | | 11; 19(23): | |
| | | | | 6579-86 | |
| E4TF1 | (GGAAGTG) | −12,−611,+650,−711, | +1174,+1347,−1381, | EMBO-J. 1994 | |
| | SEQUENCE ID NO. 24 | −717,+839,−946,+1135, | +1539,−1571,+1888, | Mar 15; 13(6): | |
| ets-related | | +1542,+2588,+2603, | −2925,−2988,+3384, | 1396-402 | |
| transcription factor | | +2732,−2799,+2813 | +3410,−3437, | | |
| binding site, | | | +3976,+4198 | | |
| possibly linked to | | | | | |
| Down's syndrome | | | | | |
| CTF NF-1 | (TTGGCT(N3)AGCCAA) | +275,−287,−603,+700, | +172,−184,−287,−449, | Cell 48:79, 1987 | |
| | SEQUENCE ID NO. 25 | −1069,+1075,−1281, | −511,+662,−1237, | | |
| RNA polymerase II | | +1547,−1704,−1834, | −1256,−1320,+1362, | | |
| recognition domain | | +2126,−2527,−2684 | −1416,+2085,−2233, | | |
| and initiation site for | | | +2242,−2349,+2722, | | |
| DNA replication | | | −2734,+3322,−3358, | | |
| | | | −3790,−4091 | | |
| CRE | (CGTCA) | | −518 | | Proc Natl Acad |
| | SEQUENCE ID NO. 26 | | | Sci USA | |
| | | | | 85:6662, 1988 | |
| CRFEII lat | (ATTGG) | −714 | −977,+1008,+1223, | Nuc Acids Res | |
| | SEQUENCE ID NO. 27 | | +1402,−1773 | 15:7761, 1987 | |
| NF-Y tk | (CCAAT) | +710 | +973,−1012,−1227, | Cell 50:863, | |
| | SEQUENCE ID NO. 28 | | −1406,+1769 | 1987 | |
| NF-Y MCHII | (ATTGG) | −714 | −977,+1008,+1223, | Proc Natl Acad | |
| | SEQUENCE ID NO. 29 | | +1402,−1773 | Sci USA | |
| | | | | 84:6249, 1987 | |
| uteroglobin | (RYYWSGTG) | +1200 | −1936,+3802,+4326 | Nuc Acids Res | |
| locus | SEQUENCE ID NO. 30 | | | 15:4535, 1987 | |
| steroid hormone | | | | | |
| receptor binding site | | | | | |
| CAAT box | (GGYCAATCT) | +572,+816,−1531, | −1229,+3580,+3896 | Nuc Acids Res | |
| | SEQUENCE ID NO. 31 | −1696,−2519,+2571 | | 14:10009, 1986 | |
| transcription | | | | | |
| element associated | | | | | |
| with RNA initiation | | | | | |
| site | | | | | |
| TATA box | (TATAWAW) | +218,−233,+454,−457, | −869,−883, +946, | Annu Rev | |
| | SEQUENCE ID NO. 32 | +521 | +1073,−1076,−1141, | Biochem 50:349, | |
| | | | −1367,+1808,−1824, | 1981 | |
| | | | −1847,−1851,+1990, | | |
| | | | −2887,+3238,−3410, | | |
| | | | −3625,−3671 | | |
| AABS | (GTGNNGYAA) | −248 | −1481 | Mol Cell Biol | |
| | SEQUENCE ID NO. 33 | | | 11:93, 1991 | |
| ATF | (WTCGTCA) | −520 | | Genetika 26:804, | |
| | SEQUENCE ID NO. 34 | | | 1990 | |
| Ad2MLP | (TATAAA) | −457 | +1073 | Cell 43:165, | |
| | SEQUENCE ID NO. 35 | | | 1985 | |
| Adh1 US2 | (CCCCGG) | +2840 | | J Biol Chem | |
| | SEQUENCE ID NO. 36 | | | 262, 7947, 1987 | |
| CuE2.1 | (CAGCTGGC) | +2646 | | Science 227:134, | |
| | SEQUENCE ID NO. 37 | | | 1985 | |
| EGR-1 | (CGCCCSCGC) | −2309 | | Nuc Acids Res | |
| | SEQUENCE ID NO. 38 | | | 20:3, 1992 | |
| ELP RS | (CAAGGTCA) | +681 | | | Mol Cell Biol |
| | SEQUENCE ID NO. 39 | | | 9:4670, 1989 | |
| GCN4 HIS3.1 | (TGACGA) | +514 | | Proc Natl Acad | |
| | SEQUENCE ID NO. 40 | | | Sci USA | |
| | | | | 83:8516, 1986 | |
| GCN4 HIS4.3 | (CAGTCA) | −2835 | | Proc Natl Acad | |
| | SEQUENCE ID NO. 41 | | | Sci USA | |
| | | | | 83:8516, 1986 | |
| GCN4 HIS4.4 | (TGACTA) | −853 | +583,−1396 | Proc Nati Acad | |
| | SEQUENCE ID NO. 42 | | | Sci USA | |
| | | | | 83:8516, 1986 | |
| GCRE | (TGACTC) | +1592,+2153,−2268 | | Cell 43:177, | |
| | SEQUENCE ID NO. 43 | | | 1985 | |
| HLA DQ beta | (ATTTGTAT) | −343 | | Nuc Acids Res | |
| | SEQUENCE ID NO. 44 | | | 15:8057, 1987 | |
| HNF5 | (TRTTTGY) | +71 | +965,+3304,+3593 | Nuc Acids Res | |
| | SEQUENCE ID NO. 45 | | | 19:131, 1991 | |
| HiNF Ahist | (AGAAATG) | +2412 | +689 | Nuc Acids Res | |
| | SEQUENCE ID NO. 46 | | | 15:1679, 1987 | |
| KROX24 | (GCGSGGGCG) | +2301 | | Proc Natl Acad | |
| | SEQUENCE ID NO. 47 | | | Sci USA | |
| | | | | 86:8737, 1989 | |
| NF-kB-consensus | (GGGRHTYYHC) | −2503,−1934 | +2726,−3966 | Cell 58:227, | |
| sequence 1 | SEQUENCE ID NO. 48 | | | 1989 | |
| pleiotrophic | | | | | |
| mediator of | | | | | |
| inducible and tissue | | | | | |
| specific gene | | | | | |
| expression | | | | | |
| MBF I | (TGCRCRC) | +1490,+1946 | −4337 | Mol Cell Biol | |
| | SEQUENCE ID NO. 49 | | | 9:5315, 1989 | |
| TFIID | (TAYAAA) | +337,−457,−566 | −999,+1073, | J Biol Chem | |
| | SEQUENCE ID NO. 50 | | −1851,+3238 | 263:12596, 1988 | |
| transcription factor | | | | | |
| lID recognition | | | | | |
| element | | | | | |
| CBP MSV | (CCAAT) | +710 | +973,−1012,−1227, Cell 44:565, | | |
| | SEQUENCE ID NO. 51 | | −1406,+1769,+2002, 1986 | | |
| | | | +2465,−2828,+3499, | | |
| | | | −3998,+4148 | | |
| CF1 | (ANATGG) | +30,+272,+757 | +368,+445,−2295, | Nuc Acids Res | |
| | SEQUENCE ID NO. 52 | | +2525,−3140,+3576,20:3, 1992 | | |
| | | | +3978 | | |
| TFIIIA | (CNGGNYNGAR) | +1845,−1886,−2076 | | +1181,−1646,−2234,Genetika 26:804, | |
| | SEQUENCE ID NO. 53 | | 4186 | 1990 | |
| transcription factor | | | | | |
| IIIA consensus | | | | | |
| binding site | | | | | |
| SV40 T-Ag | (GAGGC) | −597,+1815+2382 | +209,−1030,+1406, J Virol 46:143, | | |
| | SEQUENCE ID NO. 54 | | +1494,+3297, | 1983 | |
| tumor promoting | | | +3867+3894,+4008 | | |
| viral antigen | | | | | |
| | (TAGGC) | +36,−666,−732,−849, | 1659,3766,4102,4313 | | |
| | | −1398,−1515 | | | |
| XRE | (CACGCW) | −2152 | +2733 | Proc Natl Acad | |
| | SEQUENCE ID NO. 55 | | | Sci USA | |
| | | | | 85:5884, 1988 | |
| enhancer | (GTGGWWWG) | −273 | | Science | |
| | SEQUENCE ID NO. 56 | | | 219:626, 1983 | |
| p53 | (RRRCWWGYYY) | +445,−454,+656,−665, | +772,−781,+1736, | Nature Genetics | |
| | SEQUENCE ID NO. 57 | +1116,−1125,+1292, | −1745 | 1:45, 1992 | |
| | | −1301 | | | |
| GM-CSF | (CATTW) | −344,−536,−761,−845, | +183,−855,+985, | Mol Cell Biol | |
| | SEQUENCE ID NO. 58 | +880,−894,−1193, | −1180,+259,+876, | 10:6084, 1990 | |
| | | +1199,+1242,−2418 | +1082,−1687,−683, | | |
| | | | +956,+1094,−1841, | | |
| | | | −1980,−2322,+2880, | | |
| | | | +3603,−1997,−2396, | | |
| | | | +3002,+3616,−2165, | | |
| | | | +2441,+3404,−3723, | | |
| | | | −2309,+2675,−3580, | | |
| | | | −3982,−4053 | | |
| NF IL-6 | (TKNNGNAAK) | −247,+416,−447,−534, | −713,+1351,−1685, Nuc Acids Res | | |
| | SEQUENCE ID NO. 59 | +563,−871,−1159, | +1884,+2500,+2518,20:3, 1992 | | |
| | | −1444,+1446,+1858 | +3765,+3853,+4194 | | |
| alpha INF | (AARKGA) | +147,−250,−306,−609, | −817,+851,+910, | Cell 41:489, | |
| | SEQUENCE ID NO. 60 | +830,+890,−1238, | −1086,−1676,−1918,1985 | | |
| | | −1246,−1679,+1764, | +1993,+2161,+2532, | | |
| | | −1983,+2605 | −2597,−2884,−3006, | | |
| | | | +3412,+4165,+4208, | | |
| | | | +4265 | | |
| Octamer | (ATTTGCAT) | | +678 | Nature 329:174, | |
| | SEQUENCE ID NO. 61 | | | 1987 | |
| immunoglogin | | | | | |
| promoter element | | | | | |
| PRL | (CCTGAWWA) | | −159 | PNAS84:5211, | |
| | SEQUENCE ID NO. 62 | | | 1987 | |
| prolactin gene | | | | | |
| regulatory control | | | | | |
| element | | | | | |
| topoisomerase II | (GTNNWAYATTNATNN | | +228,−934,+1028, | Nuc Acids Res | |
| | R) | | −1106,+1835,+1839,13:1057, 1985 | | |
| alters DNA | SEQUENCE ID NO. 63 | | +1841,+2867,+3908 | | |
| supercoiling to | | | | | |
| facilitate DNA | | | | | |
| folding and | | | | | |
| replication | | | | | |
| ApoE B1 | (SCCCACCTC) | | +2322 | J Biol Chem | |
| | SEQUENCE ID NO. 64 | | | 263:8300, 1988 | |
| CATT BP | (GTCACCATT) | | +3598 | Genetika 26: | |
| | SEQUENCE ID NO. 65 | | 804, 1990 | | |
| CP1 MLP | (AACCAAT) | | +1767 | Cell 53:11, 1988 | |
| | SEQUENCE ID NO. 66 | | | | |
| CuE5 | (TGCAGGTGT) | | −3802 | Cell 56:777, | |
| | SEQUENCE ID NO. 67 | | | 1989 | |
| GAGA E74A.1 | (CTCTCTT) | | −2784 | Genetics | |
| | SEQUENCE ID NO. 68 | | | 127:535, 1991 | |
| GCN4 ILV1.2 | (TGATGT) | | +114 | Proc Natl Acad | |
| | SEQUENCE ID NO. 69 | | | Sci USA | |
| | | | | 83:8516, 1986 | |
| HSV IE | (TAATGARAT) | | +1984 | J Virol 50:708, | |
| | SEQUENCE ID NO. 70 | | | 1984 | |
| herpes virus | | | | | |
| immediate early | | | | | |
| recognition element | | | | | |
| IG kappa2 | (ATTTGCAT) | | 678 | Nuc Acids Res | |
| | SEQUENCE ID NO. 71 | | | 14:4837, 1986 | |
| IgNF A IgH, | (ATGCAAAT) | | −685 | Nature 3 19:154, | |
| | SEQUENCE ID NO. 72 | | +678 | 1986 | |
| | (ATTTGCAT) | | | | |
| MAT OCTA1 | (ATGCAAT) | | −685 | Cell 55:135, | |
| | SEQUENCE ID NO. 73 | | | 1988 | |
| MAT OCTA2 | (ATTTGCAT) | | +678 | Cell 55:135, | |
| | SEQUENCE ID NO. 74 | | | 1988 | |
| MLC1f MLC3f | (CTGAGGA) | | −3197 | Mol Cell Biol | |
| | SEQUENCE ID NO. 75 | | | 8:2581, 1988 | |
| MLTF HMGCoA | (CGTGAC) | | −2075 | Proc Natl Acad | |
| | SEQUENCE ID NO. 76 | | | Sci USA | |
| | | | | 84:3614, 1987 | |
| MTVGRE | (AGGATGT) | | −3193 | Mol Cell Biol | |
| | SEQUENCE ID NO. 77 | | | 8:3872, 1988 | |
| OBF H2B1 | (ATTTGCAT) | | +678 | Cell 50:347, | |
| | SEQUENCE ID NO. 78 | | | 1987 | |
| OBF histone | (ATTTGCAT) | | +678 | Cell 50:347, | |
| | SEQUENCE ID NO. 79 | | | 1987 | |
| OCTA 1, OCTA | (ATTTGCATNT) | | +678 | Nature 329:174, | |
| mutant | SEQUENCE ID NO. 80 | | | 1987 | |
| OCTA 3 | (ATGCAAAT) | | −685 | Proc Natl Acad | |
| | SEQUENCE ID NO. 81 | | | Sci USA | |
| | | | | 81:2650, 1984 | |
| NF E1.3 | (CTACTA) | | +586,+3205 | Genes Dev | |
| | SEQUENCE ID NO. 82 | | | 2:1089, 1988 | |
| NF E1.6 | (TATCTC) | | +1866,−3256 | Genes Dev | |
| | SEQUENCE ID NO. 83 | | | 2:1089, 1988 | |
| NF E1.5 | (TATCTC) | | −705,−2719,−2749, Genes Dev | | |
| | SEQUENCE ID NO. 84 | | +3255 | 2:1089,1988 | |

The following abbreviations are used:
R = G or A
K = G or T
B = not A (C or G or T)
V = not T (A or C or G)
Y = C or T
S = G or C
D = not C (A or G or T)
N = A or C or G or T
M = A or C
W = A or T
H = not G (A or C or T)

What is claimed:

1. An isolated nucleic acid comprising human nerve growth factor exon 1 promoter selected from 1-1786, 2274-2846, human nerve growth factor exon 3 promoter 1-1877, fragment thereof, or modified form thereof.

2. A vector comprising a nucleic acid human nerve growth factor exon 1 promoter selected from 1-1786, 2274-2846, human nerve growth factor exon 3 promoter 1-1877, fragment thereof, or modified form thereof.

3. A vector comprising pGL3-neo.

4. A nonhuman animal comprising human nerve growth factor exon 1 promoter 1-1786, 2274-2846, human nerve growth factor exon 3 promoter 1-1877, fragment thereof, or modified form thereof.

5. A method of transferring a nucleic acid to a cell comprising administering to the cell a nucleic acid encoding human nerve growth factor exon 1 promoter 1-1786, 2274-2846, human nerve growth factor exon 3 promoter 1-1877, fragment thereof, or modified form thereof.

6. A method of transferring a nucleic acid into an animal, comprising administering to the animal a nucleic acid encoding human nerve growth factor exon 1 promoter 1-1786, 2274-2846, human nerve growth factor exon 3 promoter 1-1877, fragment thereof, or modified form thereof.

7. A transformed cell comprising a nucleic acid encoding human nerve growth factor exon 1 promoter 1-1786, 2274-2846, human nerve growth factor exon 3 promoter 1-1877, fragment thereof, or modified form thereof.

8. A method of producing a protein comprising expressing a vector comprising a human nerve growth factor exon 1 promoter 1-1786, 2274-2846, human nerve growth factor exon 3 promoter 1-1877, fragment thereof, or modified form thereof operably linked to a gene encoding a protein.

9. A method of assaying a compound comprising administering a compound to a cell, wherein the cell comprises a vector which comprises a human nerve growth factor exon 1 promoter 1-1786, 2274-2846, human nerve growth factor exon 3 promoter 1-1877, fragment thereof, or modified form thereof.

10. A nonhuman transgenic animal, comprising a nucleic acid encoding human nerve growth factor exon 1 promoter 1-1786, or 2274-2846, human nerve growth factor exon 3 promoter 1-1877, fragment thereof, or modified form thereof.

11. A method for identifying a compound capable of modifying initiation of transcription of human nerve growth factor exon 1 promoter or human nerve growth factor exon 3 promoter, comprising contacting a cell comprising a nucleic acid encoding human nerve growth factor exon 1 promoter 1-1786, 2274-2846, human nerve growth factor exon 3 promoter 1-1877, fragment thereof, or modification thereof, with a compound and detecting modification of initiation of transcription.

12. A method of characterizing a compound capable of modifying initiation of transcription of human nerve growth factor exon 1 promoter or human nerve growth factor exon 3 promoter, comprising contacting a cell comprising a nucleic acid encoding human nerve growth factor exon 1 promoter 1-1786, 2274-2846, human nerve growth factor exon 3 promoter 1-1877, fragment thereof, or modification thereof, with a compound and detecting modification of initiation of transcription.

13. A compound capable of binding to a human nerve growth factor exon 1 promoter 1-1786, 2274-2846, human nerve growth factor exon 3 promoter 1-1877, fragment thereof, or modification thereof.


SEQUENCE LISTING (84 sequences)

SEQ ID NO: 1 - DNA (genomic), 21 bp; double-stranded, unknown
cttcctgggctctaatgatgc

SEQ ID NO: 2 - DNA (genomic), 21 bp; double-stranded, unknown
atagaaagctgcgtccttggc

SEQ ID NO: 3 - DNA (genomic), 22 bp; double-stranded, unknown
ggtaaaactgttattgggtccg

SEQ ID NO: 4 - DNA (genomic), 21 bp; double-stranded, unknown
ccagtgggtttccctttgacc

SEQ ID NO: 5 - DNA (genomic), 18 bp; double-stranded, unknown
tctctgctgtgccggagc

SEQ ID NO: 6 - DNA (genomic), 2846 bp; double-stranded, unknown
ggtaccactgccagcacacagtgcctggcatatggtaggctctcaatcaataatctttggagtatttttgtgtttgttgtttacatgttcttatttactcaagatccttgaagtccagggacagaaatagaggtagttaggggcagaaaggagctcttattaaatcaacatgtgcaagaagaatatgaccaacaatttagggggtgaggatggagcatataagcaaacttataatctgcttacatcacttaaagtttcccccttacataccacatggaaaagaaccacaagtgtcccaaatccttttgtccttctgaatgatgccacaagaacacatacaaatgctctgcattcaacaaccaaattctctgttattctaaaagtttaatttcatacccaaattctcaggcagctattatgtaaggcttggggctagtgctttccaaacaagtttatacatgacatgattgatggatgaattcatcctgttatctggaaattcttttgtttaattgacgatgataaatttcctaatggatcacctcgactatgatactacttttgtagaaagggccattcacggtgttccctggcctcttgccctcacttccaaagtgtgttcatacaccagcctgtatctgaacaagtcagaagtggacaagcctaaggctgggaaacaacaaggtcacaccaaagctaaggctgacttccaattccagggctttttgcctatttcatccttctcagagcatgtgtaaatggaatgaactttcttatgggagcaaacgtgaaaatagaaagaagtaagacctcaagactaatctgaatcaagggagttggaaatgcctagtcagggcttcatcttgctcaagtgccatccattaagggtaaatgaccacccccagacttaggacaggaatcatctgcttcactaaatcccagttccctggagggtgcccttctgctaagttgcactggctggtgttaccagcaatagggagattctgtgccccaccttccctccctgttactctcctcacacctacttctcctctgtggcatccatacagggtaggggtccaacccacctttgctataggaagaagcgaaggcacagacaagctcaacacgggagggagtggggctgtaaatttccaaagagctacgaatcccctggaatgctacaattaatgatgcacatttggtgacaaatttgacttcaggggtatttctcccttgctcattttatgctggggtgggaacagccctggcagaggggcaggggaaagtcaggcaagctctcctgtcaggctgaatcgagggaactcaagaaattttgaagggtcaggaagaatttgtgtggggcctggagtgtggagaggggggcatgggggcctagggtttgctggctatatcagtctggggtcacagaccccttgcaaaactgatgaaagctgcggaccttcagctcagaaaagaatattagcattgcacacagtcgcgcaaatcagcctacagtttcagaggggccaaggactccgggaagttcctggaacccagggccttaagttaaggtcccggctctagctcctgactcctgaagtcctctgccccttgtccccatgctggacttgccgggcctgggggccttctagctggttctgcagccgccttcccttgtcagaggagcttgggcacctgcccctcgcggagctccccctgggtgctcacctatcctgggataaggaaaggcgccccgaagaaaaggagcagccgatgcctggggcaccgagggcgacgccgggcagaccagggaggcactggcgaagggcaacgcgcgggggcagggcggagaggtgagggaagctgcgagcaactccgcccagccccagccagtcggcccaacgacccctgccggtgccccagaaactccccctcccggctttgcgcgcgcggcccctcagaccccagtgggtttccctttgacctctgaaggtttaaagtccttctctggctgggtctggccagccctccaggagcgatccgtctgtagtccccaggaccccctccagccgggcaccacagcccagccacagcaggtgcggggctggtggtggggaggggagggatgggggccaggatttggagcgtgtgactcaggagtacgggaggaggggctaagaattcaagaagcctgtgtgagagcagctcggcgctccggcacagcagagagcgctgggagccggaggggagcgcagcggtgagtcaggctgccccgagccgatcccgagaggggcgcagcgcgggcgcgggcaggggtggctgggcttcgcgggagagtttgcaaggataccggtctggcgagctctctggttacccccgaggctcccgcaggccgaagagcagcccggagaaatgtcccgagtgggtgtgggggcgcgggaccctcgcgggaggacgagtcggaccgagggaacagcgttagttctggtcgtggagtccctagtcccaggatggcctgcagtccagggagcagccctggcgcctgcagaagcccacggccatgccagggtctagctcgagggctagaagtggataacgcgcaagtgagggagagcgaatgggcgcggagagggatgcgccggcagctggcgcgccagggcgggaggagtggcggccagcaccgcggggggagcgcagagcgcgctggctgaggtgagcgccgagtagggaaagtgctgcgcggcccccaggtagggggaggagcggaacggggcgcgctagacctggggcagttccctcagcgcgtctcggaagggctgggagtcgtgactgagggccccggg

SEQ ID NO: 7 - DNA (genomic), 487 bp; double-stranded, unknown
caccgagggcgacgccgggcagaccagggaggcactggcgaagggcaacgcgcgggggcagggcggagaggtgagggaagctgcgagcaactccgcccagccccagccagtcggcccaacgacccctgccggtgccccagaaactccccctcccggctttgcgcgcgcggcccctcagaccccagtgggtttccctttgacctctgaaggtttaaagtccttctctggctgggtctggccagccctccaggagcgatccgtctgtagtccccaggaccccctccagccgggcaccacagcccagccacagcaggtgcggggctggtggtggggaggggagggatgggggccaggatttggagcgtgtgactcaggagtacgggaggaggggctaagaattcaagaagcctgtgtgagagcagctcggcgctccggcacagcagagagcgctgggagccggaggggagcgcagcggtgagtcaggctg

SEQ ID NO: 8 - DNA (genomic), 1786 bp; double-stranded, unknown
ggtaccactgccagcacacagtgcctggcatatggtaggctctcaatcaataatctttggagtatttttgtgtttgttgtttacatgttcttatttactcaagatccttgaagtccagggacagaaatagaggtagttaggggcagaaaggagctcttattaaatcaacatgtgcaagaagaatatgaccaacaatttagggggtgaggatggagcatataagcaaacttataatctgcttacatcacttaaagtttcccccttacataccacatggaaaagaaccacaagtgtcccaaatccttttgtccttctgaatgatgccacaagaacacatacaaatgctctgcattcaacaaccaaattctctgttattctaaaagtttaatttcatacccaaattctcaggcagctattatgtaaggcttggggctagtgctttccaaacaagtttatacatgacatgattgatggatgaattcatcctgttatctggaaattcttttgtttaattgacgatgataaatttcctaatggatcacctcgactatgatactacttttgtagaaagggccattcacggtgttccctggcctcttgccctcacttccaaagtgtgttcatacaccagcctgtatctgaacaagtcagaagtggacaagcctaaggctgggaaacaacaaggtcacaccaaagctaaggctgacttccaattccagggctttttgcctatttcatccttctcagagcatgtgtaaatggaatgaactttcttatgggagcaaacgtgaaaatagaaagaagtaagacctcaagactaatctgaatcaagggagttggaaatgcctagtcagggcttcatcttgctcaagtgccatccattaagggtaaatgaccacccccagacttaggacaggaatcatctgcttcactaaatcccagttccctggagggtgcccttctgctaagttgcactggctggtgttaccagcaatagggagattctgtgccccaccttccctccctgttactctcctcacacctacttctcctctgtggcatccatacagggtaggggtccaacccacctttgctataggaagaagcgaaggcacagacaagctcaacacgggagggagtggggctgtaaatttccaaagagctacgaatcccctggaatgctacaattaatgatgcacatttggtgacaaatttgacttcaggggtatttctcccttgctcattttatgctggggtgggaacagccctggcagaggggcaggggaaagtcaggcaagctctcctgtcaggctgaatcgagggaactcaagaaattttgaagggtcaggaagaatttgtgtggggcctggagtgtggagaggggggcatgggggcctagggtttgctggctatatcagtctggggtcacagaccccttgcaaaactgatgaaagctgcggaccttcagctcagaaaagaatattagcattgcacacagtcgcgcaaatcagcctacagtttcagaggggccaaggactccgggaagttcctggaacccagggccttaagttaaggtcccggctctagctcctgactcctgaagtcctctgccccttgtccccatgctggacttgccgggcctgggggccttctagctggttctgcagccgccttcccttgtcagaggagcttgggcacctgcccctcgcggagctccccctgggtgctcacctatcctgggataaggaaaggcgccccgaagaaaaggagcagccgatgcctgggg

SEQ ID NO: 9 - DNA (genomic), 573 bp; double-stranded, unknown
ccccgagccgatcccgagaggggcgcagcgcgggcgcgggcaggggtggctgggcttcgcgggagagtttgcaaggataccggtctggcgagctctctggttacccccgaggctcccgcaggccgaagagcagcccggagaaatgtcccgagtgggtgtgggggcgcgggaccctcgcgggaggacgagtcggaccgagggaacagcgttagttctggtcgtggagtccctagtcccaggatggcctgcagtccagggagcagccctggcgcctgcagaagcccacggccatgccagggtctagctcgagggctagaagtggataacgcgcaagtgagggagagcgaatgggcgcggagagggatgcgccggcagctggcgcgccagggcgggaggagtggcggccagcaccgcggggggagcgcagagcgcgctggctgaggtgagcgccgagtagggaaagtgctgcgcggcccccaggtagggggaggagcggaacggggcgcgctagacctggggcagttccctcagcgcgtctcggaagggctgggagtcgtgactgagggccccggg

SEQ ID NO: 10 - DNA (genomic), 34 bp; double-stranded, unknown
cagagagcgctgggagccggaggggagcgcagcg

SEQ ID NO: 11 - DNA (genomic), 4329 bp; double-stranded, unknown
aagcttcccagaagattccaagctacaaccaaagttgagaaccactgctacagaggattcagggacagtagaaagggggagccagtgaggtagacagaatgtcccacaaattctgagtgtggagggattagggggatggtgattgacagagttatcaggtttcaatagctgtggctaaggcccattagtccttgaaaaacgatcagcagaggcacagtttccttaaactatgcattgattgaattttgaacagttcgccattaatcaagtttcatggctgaaattgatcaaaatattattgattaacctcaggggtcttaaaaagaaccctctctcctctagctctaccaggctcggggttggttggacatgggttctgagatgataagtcctaggagtttggtccagaagagggaagaagcccacaacataactttggctgttatatggaaagttacattcaagcaggtggtctacagcagtggactggctctgggttggcgctttgtctttgcactggatacttcaccccatgaggaggaacaaggtggaagccctaaagcaatggttcttaaacttatgtgactatcagaatcacctgcagagctggttaaaccgcagattgttgtgtttcattcccagtttctgattcagtaggtttgtggtaaaacccaagaatttgcatttctaacatgttctaagatattactacaatactactatggaatcacacttagagaaccactgctttaaagcatgaaacccaggacagggcaagctctagaagaagtacatcagactttattaggattcctttgtgccctgtaagaaagaatagaacatgatccttaaatgagctgggatttatttccatgcatttatcaaaagtgtgagagctgatttctgtttaagtgattaccctatgaaaacagacagggttttaaaaatagatatgcatttgggttgtttgtcccaatgcctttgcattagaaatttgtaatatttaaattggatttaattttagagcctcaaccttcatcagcatgagactaaaaacaatgacaacaatatctataaaaatcatttagagtttcattattgtggacagagaatttctctctgcagtagtaaactgcttatatcaacacagaataagacaaggccaaaggcataggaaatgctggacagagtttcaaatatagcaatcagacatccagatgagattggcaggagaccctggccctggcatgcaccaaggtgacttggtccagaaattgcagatacagagccagggaatctattgtggttggcttatagtagacacccgaagaatgcagatcttcctaggaattgtggaattttttatttaaaccaaacttccctcttcttctagtcatccaaattggaggccatcctagcttgtagtggaatatccagaatatttcctgagaaagtcactattacttctctggttgctccactgattaaaagcggaggctttttgtgtcctataggaagacgttcagtgggcaggccccagaagtgggtactgcaagtctattagcacctcctgatgtgtaaggcccattctatactcctctcccctcccctactcctcttgcaatgcatggtggacctccacccagttcttgaactctggggcctttccttcccttcttccctaatgagctcctattcatccttaagaaccctgctcagatgttacctcctctatgaacatgtctctaactagtctggccagataaaaccaatttctccttccactgtgttttcatatcatgtcacatatacatcatacttatcacactgtactttaaatgtttatttatatgcatgccttttcctatctctagattacttgctttaggaagttaagtattatgtcttattctcctttgtgtccctagcacctaacacttaaaacagtggccagcacaggacctgcaagtttaagtgtttaattaatgaaataaatgaatcccaattttgggatgagagaaagcactacttaagcatctagtagcaatgcagcctggaaaacattcaaagtcacggaatctcagatgatcagagccaaaggggaccttagctgtcatctgtgccagcttcttatcctatagaggagaaagctcaaagatgaaatgaatctccttctatacaggagaagctcagagtgaactgaatcagaatgcgggtgtgtgggttccagcctgcaacctttcaggtttagccaaacacccagatgaagggtttatggactagacgaaaccatcttcccatgagtaatgggaccagataatgcccacctcttaccctggggacacgccattctccctctcccatgctaactccaaccctgggagagcatgaaaatgttctttgtcacagaatgtaaccttttaaagagtgtctgagtatgcattttcatcactagccttcaaccccaattgagtattgaaaggtttttctggtactttctggagcaagaagactattttgagcaagatgggaaaggaagaagaatggagacatcccagggcttaatttcatgatttctagtaacttgaagatcactttagaggtccttgctacctccccattctccaactcctcttcgtggttggaatttggggagcgatggtggcttttctgacatttgctttcatagcacaagctgagagggagttggatgaagatatgtggtggggatccacgctggaaaaagatatcacagggagaagatttttttgaagttgaagagagaatacggacaggaaagttaagatgtcattctagaactttattgggagggcatctccaccctacaacaaattctgtgatggacataatcattcattcatttatccgtaaatatcaccctcttgttcaaagccctccactgccttcctaatatcctgaggataaaaccatagctccttgctgtgtctctgtagacctggctcttcctggctctccagctcattttctaggtctcgttacttcatgctcagaacctttgtcttgtttctagctcagggcctttgcacttgttcttgctgcctagaatgttctctccctcattccttctcatcctccagatctcaacttgaaggccatctcctcagagctcctcgctgagcgtcctgtctacagtggcccctcgatacatcctgcagttgctctctatcatcagaccctgtaattgccttcatggcatataaagaatctggagatatcttgcttatttacacaacactgtaagctccatgagagcagaggccttgtttgtcttgtttactgctgctcagcaccaaaaacagtgcctggcacatagtcggtgcccagaaaatattgtgaatgaatgaagtgcctacatagattacattatagaagtgagaggagaatagaaaacttccattgtttctagaaactacagcctaaaattgattttttaaaattgtatcagctccatagcttccaatcctaaaatctgcctttcagtgtggtactctgagattcctgtctgattctgtgagagctccacattctctctcaaatggtcagtctgtcttatttgtcaccattactcatctgcatttttatcaaagcaccaacttgctctgaattgtcagggattttgcgtctgtataaggtattttaggctggttcagagttggatctgttatgtctgcatgtgtaatgtactgaacaatttctattttgatgccagattagggatctgctggggcaagactttggcatgtgtctagaaacacctgcactaggtgcaagatcagccatggactgtgtccaggctgaaaccaaaaggtatggcgcaagagtgagaggcaggtgccaccacaggaccatgagaggccaagctccggtaaattttggtagaccaaattctagctccttcctgggccttgatgctggtaaaatcccagaactcaaggaaatggaatttgtcctattggcacatgcctccccactgtgtagggcacagggaatgtggtgaggtacagtctaatgccagctctccccctccacagagttttggccagtggtcgtgcagtccaaggggctggatggcatgctggacccaagctcagctcagcgtccggacccaataacagttttaccaagggagcagctttctatcctggccacactgaggtaagtgcctaagggaccttggccttgccaaggtcctccctctgcagctgccagaagcaggagtcccaagtgacaggacctgagagggcaagtcagaaccaactgctgagcagcaggggcctagagaagctt

SEQ ID NO: 12 - DNA (genomic), 1877 bp; double-stranded, unknown
aagcttcccagaagattccaagctacaaccaaagttgagaaccactgctacagaggattcagggacagtagaaagggggagccagtgaggtagacagaatgtcccacaaattctgagtgtggagggattagggggatggtgattgacagagttatcaggtttcaatagctgtggctaaggcccattagtccttgaaaaacgatcagcagaggcacagtttccttaaactatgcattgattgaattttgaacagttcgccattaatcaagtttcatggctgaaattgatcaaaatattattgattaacctcaggggtcttaaaaagaaccctctctcctctagctctaccaggctcggggttggttggacatgggttctgagatgataagtcctaggagtttggtccagaagagggaagaagcccacaacataactttggctgttatatggaaagttacattcaagcaggtggtctacagcagtggactggctctgggttggcgctttgtctttgcactggatacttcaccccatgaggaggaacaaggtggaagccctaaagcaatggttcttaaacttatgtgactatcagaatcacctgcagagctggttaaaccgcagattgttgtgtttcattcccagtttctgattcagtaggtttgtggtaaaacccaagaatttgcatttctaacatgttctaagatattactacaatactactatggaatcacacttagagaaccactgctttaaagcatgaaacccaggacagggcaagctctagaagaagtacatcagactttattaggattcctttgtgccctgtaagaaagaatagaacatgatccttaaatgagctgggatttatttccatgcatttatcaaaagtgtgagagctgatttctgtttaagtgattaccctatgaaaacagacagggttttaaaaatagatatgcatttgggttgtttgtcccaatgcctttgcattagaaatttgtaatatttaaattggatttaattttagagcctcaaccttcatcagcatgagactaaaaacaatgacaacaatatctataaaaatcatttagagtttcattattgtggacagagaatttctctctgcagtagtaaactgcttatatcaacacagaataagacaaggccaaaggcataggaaatgctggacagagtttcaaatatagcaatcagacatccagatgagattggcaggagaccctggccctggcatgcaccaaggtgacttggtccagaaattgcagatacagagccagggaatctattgtggttggcttatagtagacacccgaagaatgcagatcttcctaggaattgtggaattttttatttaaaccaaacttccctcttcttctagtcatccaaattggaggccatcctagcttgtagtggaatatccagaatatttcctgagaaagtcactattacttctctggttgctccactgattaaaagcggaggctttttgtgtcctataggaagacgttcagtgggcaggccccagaagtgggtactgcaagtctattagcacctcctgatgtgtaaggcccattctatactcctctcccctcccctactcctcttgcaatgcatggtggacctccacccagttcttgaactctggggcctttccttcccttcttccctaatgagctcctattcatccttaagaaccctgctcagatgttacctcctctatgaacatgtctctaactagtctggccagataaaaccaatttctccttccactgtgttttcatatcatgtcacatatacatcatacttatcacactgtactttaaatgtttatttatatgcatgccttttcctatctctagatt

SEQ ID NO: 13 - DNA (genomic), 124 bp; double-stranded, unknown
agttttggccagtggtcgtgcagtccaaggggctggatggcatgctggacccaagctcagctcagcgtccggacccaataacagttttaccaagggagcagctttctatcctggccacactgag

SEQ ID NO: 14 - DNA (genomic), 5960 bp; double-stranded, unknown
ggtaccgagctcttacgcgtgctagcccgggctcgagatctgcgatctaagtaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccggcagtgtggttttgcaagaggaagcaaaaagcctctccacccaggcctggaatgtttccacccaatgtcgagcagtgtggttttgcaagaggaagcaaaaagcctctccacccaggcctggaatgtttccacccaatgtcgagcaaaccccgcccagcgtcttgtcattggcgaattcgaacacgcagatgcagtcggggcggcgcggtcccaggtccacttcgcatattaaggtgacgcgtgtggcctcgaacaccgagcgaccctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcggcaataaaaagacagaataaaacgcacgggtgttgggtcgtttgttcggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 15 - DNA (genomic), 8 bp; double-stranded, unknown
tttcgcgc

SEQ ID NO: 16 - DNA (genomic), 7 bp; double-stranded, unknown
tkastma

SEQ ID NO: 17 - DNA (genomic), 8 bp; double-stranded, unknown
ccscrggc

SEQ ID NO: 18 - DNA (genomic), 8 bp; double-stranded, unknown
tgtggwww

SEQ ID NO: 19 - DNA (genomic), 9 bp; double-stranded, unknown
cagctgtgg

SEQ ID NO: 20 - DNA (genomic), 10 bp; double-stranded, unknown
ctgtggaatg

SEQ ID NO: 21 - DNA (genomic), 8 bp; double-stranded, unknown
gccccacc

SEQ ID NO: 22 - DNA (genomic), 14 bp; double-stranded, unknown
gggaaatagaaast

SEQ ID NO: 23 - DNA (genomic), 8 bp; double-stranded, unknown
tgggaatt

SEQ ID NO: 24 - DNA (genomic), 7 bp; double-stranded, unknown
ggaagtg

SEQ ID NO: 25 - DNA (genomic), 15 bp; double-stranded, unknown
ttggctnnnagccaa

SEQ ID NO: 26 - DNA (genomic), 5 bp; double-stranded, unknown
cgtca

SEQ ID NO: 27 - DNA (genomic), 5 bp; double-stranded, unknown
attgg

SEQ ID NO: 28 - DNA (genomic), 5 bp; double-stranded, unknown
ccaat

SEQ ID NO: 29 - DNA (genomic), 5 bp; double-stranded, unknown
attgg

SEQ ID NO: 30 - DNA (genomic), 8 bp; double-stranded, unknown
ryywsgtg

SEQ ID NO: 31 - DNA (genomic), 9 bp; double-stranded, unknown
ggycaatct

SEQ ID NO: 32 - DNA (genomic), 7 bp; double-stranded, unknown
tatawaw

SEQ ID NO: 33 - DNA (genomic), 9 bp; double-stranded, unknown
gtgnngyaa

SEQ ID NO: 34 - DNA (genomic), 7 bp; double-stranded, unknown
wtcgtca

SEQ ID NO: 35 - DNA (genomic), 6 bp; double-stranded, unknown
tataaa

SEQ ID NO: 36 - DNA (genomic), 6 bp; double-stranded, unknown
ccccgg

SEQ ID NO: 37 - DNA (genomic), 8 bp; double-stranded, unknown
cagctggc

SEQ ID NO: 38 - DNA (genomic), 9 bp; double-stranded, unknown
cgcccscgc

SEQ ID NO: 39 - DNA (genomic), 8 bp; double-stranded, unknown
caaggtca

SEQ ID NO: 40 - DNA (genomic), 6 bp; double-stranded, unknown
tgacga

SEQ ID NO: 41 - DNA (genomic), 6 bp; double-stranded, unknown
cagtca

SEQ ID NO: 42 - DNA (genomic), 6 bp; double-stranded, unknown
tgacta

SEQ ID NO: 43 - DNA (genomic), 6 bp; double-stranded, unknown
tgactc

SEQ ID NO: 44 - DNA (genomic), 8 bp; double-stranded, unknown
atttgtat

SEQ ID NO: 45 - DNA (genomic), 7 bp; double-stranded, unknown
trtttgy

SEQ ID NO: 46 - DNA (genomic), 7 bp; double-stranded, unknown
agaaatg

SEQ ID NO: 47 - DNA (genomic), 9 bp; double-stranded, unknown
gcgsgggcg

SEQ ID NO: 48 - DNA (genomic), 10 bp; double-stranded, unknown
gggrhtyyhc

SEQ ID NO: 49 - DNA (genomic), 7 bp; double-stranded, unknown
tgcrcrc

SEQ ID NO: 50 - DNA (genomic), 6 bp; double-stranded, unknown
tayaaa

SEQ ID NO: 51 - DNA (genomic), 5 bp; double-stranded, unknown
ccaat

SEQ ID NO: 52 - DNA (genomic), 6 bp; double-stranded, unknown
anatgg

SEQ ID NO: 53 - DNA (genomic), 10 bp; double-stranded, unknown
cnggnyngar

SEQ ID NO: 54 - DNA (genomic), 5 bp; double-stranded, unknown
gaggc

SEQ ID NO: 55 - DNA (genomic), 6 bp; double-stranded, unknown
cacgcw

SEQ ID NO: 56 - DNA (genomic), 8 bp; double-stranded, unknown
gtggwwwg

SEQ ID NO: 57 - DNA (genomic), 10 bp; double-stranded, unknown
rrrcwwgyyy

SEQ ID NO: 58 - DNA (genomic), 5 bp; double-stranded, unknown
cattw

SEQ ID NO: 59 - DNA (genomic), 9 bp; double-stranded, unknown
tknngnaak

SEQ ID NO: 60 - DNA (genomic), 6 bp; double-stranded, unknown
aarkga

SEQ ID NO: 61 - DNA (genomic), 8 bp; double-stranded, unknown
atttgcat

SEQ ID NO: 62 - DNA (genomic), 8 bp; double-stranded, unknown
cctgawwa

SEQ ID NO: 63 - DNA (genomic), 16 bp; double-stranded, unknown
gtnnwayattnatnnr

SEQ ID NO: 64 - DNA (genomic), 9 bp; double-stranded, unknown
scccacctc

SEQ ID NO: 65 - DNA (genomic), 9 bp; double-stranded, unknown
gtcaccatt

SEQ ID NO: 66 - DNA (genomic), 7 bp; double-stranded, unknown
aaccaat

SEQ ID NO: 67 - DNA (genomic), 9 bp; double-stranded, unknown
tgcaggtgt

SEQ ID NO: 68 - DNA (genomic), 7 bp; double-stranded, unknown
ctctctt

SEQ ID NO: 69 - DNA (genomic), 6 bp; double-stranded, unknown
tgatgt

SEQ ID NO: 70 - DNA (genomic), 9 bp; double-stranded, unknown
taatgarat

SEQ ID NO: 71 - DNA (genomic), 8 bp; double-stranded, unknown
atttgcat

SEQ ID NO: 72 - DNA (genomic), 8 bp; double-stranded, unknown
atgcaaat

SEQ ID NO: 73 - DNA (genomic), 7 bp; double-stranded, unknown
atgcaat

SEQ ID NO: 74 - DNA (genomic), 8 bp; double-stranded, unknown
atttgcat

SEQ ID NO: 75 - DNA (genomic), 7 bp; double-stranded, unknown
ctgagga

SEQ ID NO: 76 - DNA (genomic), 6 bp; double-stranded, unknown
cgtgac

SEQ ID NO: 77 - DNA (genomic), 7 bp; double-stranded, unknown
aggatgt

SEQ ID NO: 78 - DNA (genomic), 8 bp; double-stranded, unknown
atttgcat

SEQ ID NO: 79 - DNA (genomic), 8 bp; double-stranded, unknown
atttgcat

SEQ ID NO: 80 - DNA (genomic), 10 bp; double-stranded, unknown
atttgcatnt

SEQ ID NO: 81 - DNA (genomic), 8 bp; double-stranded, unknown
atgcaaat

SEQ ID NO: 82 - DNA (genomic), 6 bp; double-stranded, unknown
ctacta

SEQ ID NO: 83 - DNA (genomic), 6 bp; double-stranded, unknown
tatctc

SEQ ID NO: 84 - DNA (genomic), 6 bp; double-stranded, unknown
tatctc